(19) United States
(12) Patent Application Publication
(10) Pub. No.: US 2026/0263212 A1
(43) Pub. Date: Sep. 10, 2026

(54) PROSTHETIC CARDIAC VALVE DEVICE, SYSTEMS, AND METHODS

(71) Applicant: Shifamed Holdings, LLC, Campbell, CA (US)

(72) Inventors: Jasper Ellington ADAMEK-BOWERS, San Francisco, CA (US); Alice YANG, Campbell, CA (US); Ryan William BOYD, Santa Cruz, CA (US); Mitchell YOUNG, San Francisco, CA (US); Sarah Louise GARCON, Milpitas, CA (US); Timothy John CONNEELY, Palo Alto, CA (US); Kathryn GREENBERG, Santa Clara, CA (US); Jordan SKARO, San Jose, CA (US); Jonathan Hwan OAKDEN, San Jose, CA (US); Ali SALAHIEH, Saratoga, CA (US); Savina BRACHTHAUSER BALCELLS, Los Gatos, CA (US); Noah GOLDSMITH, Santa Cruz, CA (US)

(21) Appl. No.: 19/162,907

(22) PCT Filed: Mar. 7, 2024
(86) PCT No.: PCT/US2024/018923

§ 371 Date: Sep. 5, 2025

Related U.S. Application Data
(60) Provisional application No. 63/488,945, filed on Mar. 7, 2023.
(60) Provisional application No. 63/590,364, filed on Oct. 13, 2023.

(51) Int. Cl. A61F 2/24

(52) U.S. Cl. CPC A61F 2/2409; A61F 2/2418; A61F 2220/0008; A61F 2230/0091

(57) ABSTRACT

Prosthetic heart valves and delivery systems and methods of delivery and use are provided herein. The heart valve can include a valve frame with a flared atrial portion, a flared ventricular portion, and a narrowed central waist portion between the flared atrial and ventricular portions. The system can further include a helical anchoring device configured to retain the valve frame within the native mitral valve anatomy. The valve frame and anchoring device are configured to dynamically expand, and contract once implanted in response to the dynamic heart cycle. When the anchor and frame are in an at-rest state in which low heart pressures are applied against the system, the anchor applies very low retaining forces against the frame. When heart pressures increase and cause axial movement of the frame relative to the anchor, the retaining forces applied by the anchoring device to the frame increase.

### PRIORITY CLAIM

This patent application claims priority to U.S. Provisional Patent Application No. 63/488,945, titled “PROSTHETIC CARDIAC VALVE DEVICE, SYSTEMS, AND METHODS,” filed on Mar. 7, 2023, and U.S. Provisional Patent Application No. 63/590,364, titled “PROSTHETIC CARDIAC VALVE DEVICE, SYSTEMS, AND METHODS,” filed Oct. 13, 2023, each of which are herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Heart disease is leading cause of death in the U.S. and worldwide. Valvular heart disease is a major component of heart failure, with stenosis and regurgitation being effects often present in those suffering from valvular disease. Blood flow between heart chambers is regulated by native valves-the mitral valve, the aortic valve, the pulmonary valve, and the tricuspid valve. Patients with valvular disease have abnormal anatomy and/or function of at least one valve. For example, a valve may suffer from insufficiency, also referred to as regurgitation, when the valve does not fully close, thereby allowing blood to flow retrograde from the left ventricle into the left atrium. Valve stenosis can cause a valve to fail to open properly, requiring higher pressures to push blood into the left ventricle. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. The mitral valve is circumscribed by the mitral annulus, which provides the insertion site for anterior and posterior leaflets of the mitral valve. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve replacement devices, for example, is that the mitral valve devices are too large in diameter to be delivered transeptally, requiring transapical access instead. Furthermore, existing mitral valve replacement devices are not optimized with respect to strength-weight ratio and often take up too much space within the valve chambers, resulting in obstruction of outflow from the ventricle into the aorta and/or thrombosis. With transcatheter edge-to-edge repair (TEER) and other repair techniques, the patients often still experience some level of regurgitation such that the underlying disease is not completely addressed. Other treatments are not minimally invasive and thus not suitable for older patients.

Many total mitral valve replacement (TMVR) systems are designed based on transaortic valve implantation (TAVI) systems and have long, bulky valves, which can extend into the left ventricular outflow tract (LVOT) and negatively impact hemodynamics (the implanted prosthetic valve forming a so-called “neo LVOT”). Estimates have approximately 50% of heart failure patients being screened out of TMVR based on left ventricular outflow tract obstruction (LVOTO), with some estimates increasing that to more than 75%. Also, TMVR valves which have origins with TAVI designs are difficult to implant in the mitral position. Since TAVI valves are designed to sit in the blood vessel-like aorta, their designs are typically based on stent-like structures that are supported (e.g., anchored) along their length by surrounding tissue. In contrast, the mitral valve sits between two chambers, the left atrium and left ventricle. TMVR designs based on TAVI valves intended for implantation in a vessel do not fully address the challenges of implanting a prosthetic valve that is between two chambers. These challenges include designs that account for the angle of the prosthetic valve with respect to both mitral and aortic valves, and the length of the prosthetic valve in the mitral anatomy—the failure to address these challenges resulting in high prevalence of LVOTO.

Patients suffering from valve insufficiency frequently have larger-than-typical native valve sizes. As described in prior publications (such as US 2009/0319037 to Rowe), a large prosthetic valve has been considered necessary to treat patients with enlarged hearts. Percutaneous delivery of such a large valve has been understood to be of considerable difficulty. Further, self-expanding (in contrast to balloon expandable) valves were thought to be inappropriate to implant into some patients, as concerns existed that prior self-expanding prosthetic valve designs exhibited chronic outward force on the native tissue once implanted. Chronic outward force against certain tissues, especially the annulus, can result in deleterious remodeling that in some cases can exacerbate the underlying conditions for the patient (e.g., regurgitation). The '037 publication described as preferred approaches implantation of a balloon-expandable prosthesis that in one case would be anchored directly to native tissue, and in another case would utilize a closed support band of fixed diameter that would circumscribe and frictionally secure native tissue in the sub-annular space to the valve stent (the stent being balloon-expanded into the support band). The support band is delivered via a trans-aortic or trans-apical approach by advancement over prior-placed loops via the same approach, while the prosthetic valve is typically delivered trans-apical. US 2013/0116779 to Weber provides a later example of a similar two-part system with an outer device (support structure) that is delivered over prior-placed loop(s) (both via trans-aortic approach), with the prosthetic valve delivery transeptally. The above two-part systems require separate access points and delivery paths in the patient, increasing both procedural complexity and closure risk for the patient. Furthermore, support structure delivery apparatus that extends from the sub-mitral space and back through the aorta during the valve delivery presents risks during the valve deployment procedure. For example, manipulation of the valve delivery system can transmit forces onto the support structure and, via its delivery apparatus, the aortic valve and/or aorta-increasing the risk of initiating aortic regurgitation (AR) in the patient (e.g., torrential AR). The support structure delivery apparatus may similarly interfere with the valve delivery system, and in some cases such interference may lead to mitral regurgitation (MR) (e.g., torrential MR).

Other approaches to atrio-ventricular (A-V) valve replacement derive via adaptation from related A-V procedures. For example, a number of approaches and devices have been adapted from those that were initially developed for annuloplasty of the diseased valve. These approaches rely upon anchoring a support structure and modifying the tissue adjacent the annulus of the native valve to reduce the size thereof, which can be accomplished via suture or other tissue penetrating approach, or clamping of the valve annulus. Examples of such approaches include U.S. Pat. No. 6,419,696 to Ortiz, US 2006/0195134 to Crittenden, WO 2008/058940 to Keranen, US 2010/0076549 to Keidar, and US 2012/0016464 to Seguin. Such devices include a first portion of the support device placed on a first side of the valve (e.g., left atrium), and a second portion of the support device placed on a second side of the valve (e.g., left ventricle), with the tissue clamped (axially, superior-to-inferior) between the first and second portions. A-V replacement valves that are designed to cooperate with such support structures must accommodate the support structure maintaining these tissue interactions with the native valve. Such replacement systems generally require distinct sealing elements on one or both of the support structure and prosthetic valve to address paravalvular leakage (PVL), which adds to device construction-and delivery complexity.

Thus, a new valve device that overcomes some or all of these deficiencies is desired.

### SUMMARY OF THE DISCLOSURE

A mitral valve replacement system is provided, comprising a helical anchoring device; and a heart valve replacement frame configured to self-expand within the helical anchoring device; wherein self-expansion of the heart valve replacement frame into the helical anchoring device is configured to reduce a number of turns of the helical anchoring device.

In some aspects, self-expansion of the mitral valve replacement frame into the helical anchoring device reduces a number of turns of the helical anchoring device by up to 30%.

In another aspect, self-expansion of the mitral valve replacement frame into the helical anchoring device reduces a number of turns of the helical anchoring device by 20% to 30%.

In some aspects, the helical anchoring device comprises approximately 1.6 to 1.8 turns when it is deployed within the left ventricle.

In other aspects, the helical anchoring device comprises approximately 1.1 to 1.25 turns when the mitral valve replacement frame is self-expanded within the helical anchoring device.

In some aspects, the helical anchoring device is configured to be deployed entirely in a left ventricle of a human subject.

In other aspects, the heart valve replacement frame comprises a flared atrial portion configured to engage atrial tissue of the subject, a narrowed central portion configured to engage the helical anchoring device, and a flared ventricular portion configured to extend into the left ventricle of the subject.

In one aspect, a percentage of the helical anchor that overlaps with an adjacent turn of the helical anchor is reduced when the heart valve replacement frame is self-expanded into the helical anchoring device.

In some aspects, the heart valve replacement frame comprises a flared ventricular portion, a central waist portion, and a flared atrial portion.

In one aspect, the helical anchoring device is configured to reside within the central waist portion of the heart valve replacement frame when the heart valve replacement frame is self-expanded into the helical anchoring device

A mitral valve replacement system is provided, comprising: a helical anchoring device comprising configured to be disposed entirely within a left ventricle of a subject; and a self-expandable mitral valve replacement frame comprising a flared atrial portion configured to engage atrial tissue of the subject, a narrowed central portion configured to engage the helical anchoring device, and a flared ventricular portion configured to extend into the left ventricle of the subject; wherein self-expansion of the mitral valve replacement frame into the helical anchoring device is configured to reduce a number of turns of the helical anchoring device.

In some aspects, self-expansion of the mitral valve replacement frame into the helical anchoring device reduces a number of turns of the helical anchoring device by up to 30%.

In other aspects, self-expansion of the mitral valve replacement frame into the helical anchoring device reduces a number of turns of the helical anchoring device by 20% to 30%.

In some aspects, the helical anchoring device comprises approximately 1.6 to 1.8 turns when it is deployed within the left ventricle.

In other aspects, the helical anchoring device comprises approximately 1.1 to 1.25 turns when the mitral valve replacement frame is self-expanded within the helical anchoring device.

In additional aspects, the helical anchoring device is configured to be deployed entirely in a left ventricle of a human subject.

In some aspects, the heart valve replacement frame comprises a flared atrial portion configured to engage atrial tissue of the subject, a narrowed central portion configured to engage the helical anchoring device, and a flared ventricular portion configured to extend into the left ventricle of the subject.

In other aspects, a percentage of the helical anchor that overlaps with an adjacent turn of the helical anchor is reduced when the heart valve replacement frame is self-expanded into the helical anchoring device.

In some aspects, the helical anchoring device is configured to apply a retention force less than 4 lbs. to the valve replacement frame in an at-rest state in which the helical anchoring device resides within the narrowed central portion.

A method is provided, comprising: deploying a helical anchoring device entirely in a left ventricle of a subject; advancing a mitral valve replacement frame through the helical anchoring device; and self-expanding the mitral valve replacement device within the helical anchoring device to reduce a number of turns of the helical anchoring device.

In some aspects, self-expanding the mitral valve replacement device within the helical anchoring device reduces the number of turns of the helical anchoring device by up to 30%.

In other aspects, self-expanding the mitral valve replacement device within the helical anchoring device reduces the number of turns of the helical anchoring device by up to 20%.

In some aspects, self-expanding the mitral valve replacement device within the helical anchoring device reduces the number of turns of the helical anchoring device by 20% to 30%.

In some aspects, self-expanding the mitral valve replacement device within the helical anchoring device reduces the number of turns of the helical anchoring from approximately 1.6 to 1.8 turns to approximately 1.1 to 1.25 turns.

A mitral valve replacement system is provided, comprising: a helical anchoring device; and a heart valve replacement frame configured to self-expand within the helical anchoring device; wherein self-expansion of the heart valve replacement frame into the helical anchoring device is configured to increase a diameter of the helical anchoring device.

In some aspects, self-expansion of the mitral valve replacement frame into the helical anchoring device increases the diameter of the helical anchoring device by up to 25%.

In other aspects, self-expansion of the mitral valve replacement frame into the helical anchoring device increases the diameter of the helical anchoring device by 20% to 30%.

In some aspects, self-expansion of the mitral valve replacement frame into the helical anchoring device increases the diameter from approximately 23 mm to approximately 28 mm.

In other aspects, the helical anchoring device is configured to be deployed entirely in a left ventricle of a human subject.

In one aspect, the heart valve replacement frame comprises a flared atrial portion configured to engage atrial tissue of the subject, a narrowed central portion configured to engage the helical anchoring device, and a flared ventricular portion configured to extend into the left ventricle of the subject.

In additional aspects, a percentage of the helical anchor that overlaps with an adjacent turn of the helical anchor is reduced when the heart valve replacement frame is self-expanded into the helical anchoring device.

A mitral valve replacement system is provided, comprising: a helical anchoring device comprising configured to be disposed entirely within a left ventricle of a subject; and a self-expandable mitral valve replacement frame comprising a flared atrial portion configured to engage atrial tissue of the subject, a narrowed central portion configured to engage the helical anchoring device, and a flared ventricular portion configured to extend into the left ventricle of the subject; wherein self-expansion of the heart valve replacement frame into the helical anchoring device is configured to increase a diameter of the helical anchoring device.

A method is provided, comprising: deploying a helical anchoring device entirely in a left ventricle of a subject; advancing a mitral valve replacement frame through the helical anchoring device; and self-expanding the mitral valve replacement device within the helical anchoring device to increase a diameter of the helical anchoring device.

In some aspects, self-expansion of the mitral valve replacement frame into the helical anchoring device increases the diameter of the helical anchoring device by up to 25%.

In other aspects, self-expansion of the mitral valve replacement frame into the helical anchoring device increases the diameter of the helical anchoring device by 20% to 30%.

In some aspects, self-expansion of the mitral valve replacement frame into the helical anchoring device increases the diameter from approximately 23 mm to approximately 28 mm.

A mitral valve replacement system is provided, comprising: a helical anchoring device; and a heart valve replacement frame configured to self-expand within the helical anchoring device; wherein self-expansion of the heart valve replacement frame into the helical anchoring device is configured to increase a diameter of the helical anchoring device and reduce a number of turns of the helical anchoring device.

A mitral valve replacement system is provided, comprising: a helical anchoring device comprising configured to be disposed entirely within a left ventricle of a subject; and a self-expandable mitral valve replacement frame comprising a flared atrial portion configured to engage atrial tissue of the subject, a narrowed central portion configured to engage the helical anchoring device, and a flared ventricular portion configured to extend into the left ventricle of the subject; wherein self-expansion of the heart valve replacement frame into the helical anchoring device is configured to increase a diameter of the helical anchoring device and reduce a number of turns of the helical anchoring device.

A method is provided, comprising: deploying a helical anchoring device entirely in a left ventricle of a subject; advancing a mitral valve replacement frame through the helical anchoring device; and self-expanding the mitral valve replacement device within the helical anchoring device to increase a diameter of the helical anchoring device and reduce a number of turns of the helical anchoring device.

A method is provided, comprising: deploying a helical anchoring device entirely in a left ventricle of a subject; advancing a mitral valve replacement frame through the helical anchoring device; and self-expanding the mitral valve replacement device within the helical anchoring device to adjust an axial position thereof with respect to the replacement frame.

In some aspects, self-expanding the mitral valve replacement device increases a diameter of the helical anchoring device.

A method is provided, comprising: deploying a helical anchoring device entirely in a left ventricle of a subject, the helical anchoring device having an anchor rest state; advancing a mitral valve replacement frame in a compressed state through the helical anchoring device; and expanding the mitral valve replacement frame via self-expansion from the compressed state toward a frame rest state, the mitral valve replacement frame (further) expanding the helical anchor to a diameter beyond its anchor rest state.

In some aspects, expanding the mitral valve replacement frame changes an axial position of the anchor with respect to the frame.

In one aspect, axial movement between frame and anchor is limited to bottom half (ventricular flare/waist).

In some aspects, axial movement between the frame and the anchor moves the anchor toward the central waist portion.

In other aspects, further expansion of the anchor occurs once anchor has first moved axially toward the central waist portion.

A mitral valve replacement system is provided, comprising: an anchoring device configured to be deployed entirely in a left ventricle of a human subject; and a mitral valve replacement frame configured to self-expand within the anchoring device, the mitral valve replacement frame having a flared atrial portion, a narrowed central waist portion, and a flared ventricular portion; wherein the anchoring device is configured to apply a retention force no larger than 1.5 lbs. to the mitral valve replacement frame when the anchoring device resides within the narrowed central waist portion to retain the mitral valve replacement frame within a mitral annulus of the human subject.

In some aspects, the anchoring device is configured to encircle chordae in the left ventricle.

In other aspects, the anchoring device comprises a helical shape with less than two turns.

In additional aspects, comprising a plurality of leaflets coupled to the mitral valve replacement frame.

In some aspects, the anchoring device comprises an at-rest configuration with an at-rest diameter, wherein the anchoring device expands to a delivery configuration with a delivery diameter that is larger than the at-rest diameter when the mitral valve replacement frame is expanded within the anchoring device.

In one aspect, the delivery diameter is 20-25% larger than the at-rest diameter.

A method is provided, comprising: deploying an anchoring device entirely in a left ventricle of a subject; advancing a mitral valve replacement frame through the anchoring device; and self-expanding the mitral valve replacement device within the anchoring device, wherein the anchoring device is configured to apply a retention force no larger than 1.5 lbs to the mitral valve replacement frame when the anchoring device resides within a narrowed central waist portion of the mitral valve replacement frame to retain the mitral valve replacement frame within a mitral annulus of the subject.

In some aspects, the method includes applying retention forces larger than 1.5 lbs. to the mitral valve replacement frame when pressures applied by the left ventricle to the mitral valve replacement frame urge the mitral valve replacement frame axially with respect to the anchor.

In other aspects, the pressures urge the mitral valve replacement frame axially to position the anchor along or within a ventricular flared portion of the mitral valve replacement frame.

In one aspect, the pressures urge the mitral valve replacement frame axially to position the anchor along or within an atrial flared portion of the mitral valve replacement frame.

A mitral valve replacement system is provided, comprising: an anchoring device configured to be deployed entirely in a left ventricle of a human subject; and a mitral valve replacement frame configured to self-expand within the anchoring device; wherein the anchoring device is configured to apply a retention force of 4 lbs. or less to the mitral valve replacement frame when the anchoring device resides within a narrowed central waist portion of the mitral valve replacement frame to retain the mitral valve replacement frame within a mitral annulus of the human subject.

In one aspect, the anchoring device is configured to apply retention forces larger than 1.5 lbs. to the mitral valve replacement frame when pressures applied by the left ventricle to the mitral valve replacement frame urge the mitral valve replacement frame axially with respect to the anchor.

In some aspects, the pressures urge the mitral valve replacement frame axially to position the anchor along or within a ventricular flared portion of the mitral valve replacement frame.

In other aspects, the pressures urge the mitral valve replacement frame axially to position the anchor along or within an atrial flared portion of the mitral valve replacement frame.

A mitral valve replacement system is provided, comprising: a helical anchoring device comprising more than 1 turn (or optionally more than 360 degrees), the helical anchoring device being configured to be deployed around chordae entirely in a left ventricle of a subject; and a self-expandable mitral valve replacement frame comprising a flared atrial portion configured to engage atrial tissue of the subject, a narrowed central portion configured to engage the helical anchoring device, and a flared ventricular portion configured to extend into the left ventricle of the subject; wherein a diameter of the anchoring device is configured to increase and the anchoring device is configured to apply a retention force of less than 4 lbs. to the mitral valve replacement frame when the mitral valve replacement frame is self-expanded within the anchoring device.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows an exemplary prosthetic mitral valve in place in a patient's heart.

FIG. 2 shows aspects of an anchor delivery subsystem having a proximal controller and three nested catheters.

FIG. 3 is a schematic cross-sectional representation of select components of an anchor control catheter.

FIGS. 4-7 are embodiments of guide arm and the distal end of rotation control shaft.

FIG. 8 is a sample cross-sectional image of the guide arm from FIGS. 4-5.

FIG. 9 is a sample cross-sectional image of the guide arm from FIGS. 6-7.

FIGS. 10A, 10B and 11 illustrate a support structure of an intermediate part of a guide arm.

FIG. 12 shows the guide arm in a self-assembly position in which the anchor within the guide arm is fully axially extended.

FIGS. 13-14 show the guide arm in an encircling position in which the anchor is retracted within the guide arm.

FIGS. 15A-15I show details of an anchor.

FIG. 16 shows aspects of a valve delivery subsystem having a proximal controller and three nested catheters.

FIG. 17 shows a view of the distal end of the valve delivery subsystem.

FIGS. 18A-18B show embodiments of a valve prosthesis having a valve frame structure.

FIGS. 19A-19Y illustrate systems and methods for implanting a prosthetic mitral valve and anchor in a heart of a subject.

### DETAILED DESCRIPTION

Described herein are systems, devices, and methods for treatment or replacement of a diseased native valve of the heart, such as the mitral valve. The systems, devices, and methods described herein provide for a consistent, forgiving delivery of a prosthetic valve system (anchor and valve), the implanted prosthetic valve system purpose-built for the mitral anatomy and addressing native valve regurgitation (e.g., PVL) while limiting or avoiding (e.g., neo) LVOTO in the patient. The anchor is formed of one or more loops in an open-ended (e.g., having free ends) configuration. When the prosthetic valve is implanted therein, the anchor can accommodate (self-) expansion to permit the prosthetic valve to fully self-expand to its designed dimensions. The valve comprises a waist sized and shaped to receive the anchor, along with atrial and ventricular flared ends. The atrial portion of the prosthetic valve is designed to be sufficiently soft so as to 1) atraumatically conform to the native tissue of the atrium and prevent PVL, while being sufficiently stiff so as to 2) promote full self-expansion of the prosthetic valve, and to 3) secure the prosthetic valve to the native tissue (in cooperation with the anchor that resides fully in the subvalvular space). A two-stage implantation procedure (anchor, followed by prosthetic valve) enables a true 28 French (Fr) delivery system, with the anchor being fully delivered (e.g., untethered) prior to the prosthetic valve delivery. The prosthetic valve design is “self-centering,” enabling a forgiving delivery into the prior-placed anchor that is tolerant to off-axis alignment thereto by as much as 45 degrees for achieving proper valve positioning upon implantation. With proper positioning, the seating of the prosthetic valve in the atrium provides an advantageous angle with respect to the (neo) LVOT, which in combination with its axial height (from atrial end to ventricular end) greatly reduces (or eliminates) incidence of LVOTO. The purpose-built prosthetic valve system described herein expands the population of patients for whom TMVR can be made a viable option, as well as reducing procedural complexity for clinicians.

This disclosure is directed to replacement mitral valve systems and methods. In general, described herein is a replacement prosthesis that can include a valve frame and a separate anchor disposed therearound. This disclosure is further directed to delivery systems for implanting a mitral valve replacement device within a mitral valve of a patient. The delivery systems disclosed herein can include one or more steerable or controllable catheters for implanting the prosthetic heart valve frame and the anchor. The anchor is adapted to be disposed in a ventricle adjacent a native valve of a patient's heart and the valve frame supporting prosthetic valve leaflets is adapted to be delivered after delivery of the anchor and then self-expanded within the anchor. In particular, the valve is a prosthetic mitral valve, and the delivery system of this invention delivers the valve's two components transeptally. In use, the delivery system advances distally from an entry point in the patient's femoral vein, enters the right atrium of the heart, and passes through the septum into the left atrium during the implantation of the anchor, and the valve frame is then expanded inside the anchor.

Because the anatomy of the heart may differ from patient to patient, it may be desirable to be able to control the movement, position, and/or orientation of the delivery system while delivering and implanting the anchor and the valve frame. It may also be necessary to retrieve the anchor and/or the valve during implantation if their position is not quite right. The prosthetic valve delivery system of this invention therefore provides mechanisms for navigating the anchor and the valve and for controllably releasing the anchor and the valve when they have been correctly placed.

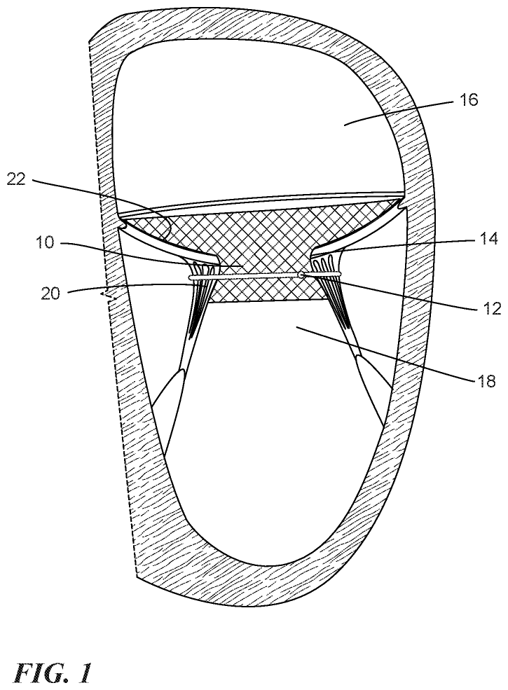

FIG. 1 shows an exemplary prosthetic mitral valve 10 in place in a patient's heart. Valve 10 includes an anchor 12 and a valve frame 14. Moveable leaflets (not shown) attached to the valve frame take the place, and perform the function, of the native valve leaflets. As shown, anchor 12 has been placed around chordae tendinae (“chordae” or “chords”) 20 and/or portions of native leaflets in the left ventricle 18. Valve frame 14 extends between the left atrium 16 and the left ventricle 18 through the native valve annulus 22.

The anchor 12 and valve frame 14 of valve 10 are implanted separately. Anchor 12 is delivered first and placed around the chordae 20. Valve frame 14 is thereafter delivered and expanded within anchor 12. In order to advance the valve components from an opening in the patient's groin to the heart, the delivery system might need to be pushed, bent, and/or rotated to navigate the anatomy of the intervening vasculature.

Because the anchor and the frame are delivered separately, the delivery system described herein has two main subsystems: an anchor delivery subsystem and a valve frame delivery subsystem.

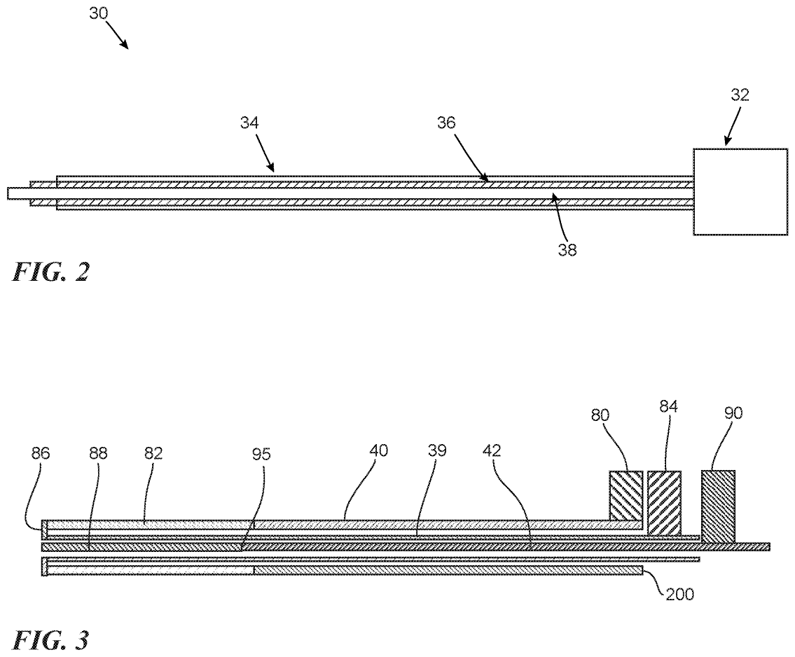

FIG. 2 shows aspects of an anchor delivery subsystem 30 having a proximal controller 32 and three nested catheters: An outer steering catheter 34, an inner steering catheter 36 movably disposed within the lumen of the outer steering catheter 34, and an anchor control catheter 38 movably disposed within the lumen of the inner steering catheter. A guide arm (not shown in FIG. 2) extends from a distal end of the anchor control catheter, as described below. Also not shown is a tether releasably connected at its distal end to an anchor (not shown) movably disposed within the inner steering catheter 36. Additional details on the nested catheters of the anchor delivery subsystem are found in co-pending US Provisional Application No. 63/366,115, filed Jun. 9, 2022, which is incorporated herein by reference. Outer steering catheter 34, inner steering catheter 36, anchor control catheter 38, and the tether are all operatively connected to the proximal controller 32. While the proximal controller 32 is shown as a single component for ease of illustration, it should be understood that each of the nested catheters can include their own proximal controllers. An introducer sheath (not shown) may be used to introduce the three nested catheters into the patient's vasculature. The introducer sheath can gain access to the left atrium over a guidewire (not shown).

FIG. 3 is a schematic cross-sectional representation of the major components of the anchor control catheter 38. A rotation control shaft 40 of the anchor control catheter 38 extends distally from an actuator 80 in a proximal controller. A guide arm portion 82 extends distally from the rotation control shaft 40.

Guide arm 82 has active and passive features that enable it to assemble into a (e.g., tapered) spiral in the left atrium, as described below. Specifically, guide arm 82 has shape set features and cut pattern features that enable the guide arm to be flexible when within the steerable catheters. When it emerges from the inner steerable catheter, however, guide arm 82 achieves a desired shape through a combination of the shape set features, actuation of the cut pattern, and selected positioning of the anchor therein, to hold the desired shape.

Actuation catheter 39 extends through the lumen of the rotation control shaft 40 and guide arm 82 from an actuator 84 in the proximal controller to the distal end of the guide arm 82. A cap 86 extends over, and is attached to, the distal end of guide arm 82. The cap is configured to couple the distal end of the actuation catheter 39, distal end of the guide arm 82, and the outer jacket into a smooth and atraumatic distal tip. Proximal movement of actuator 84 places guide arm 82 and rotation control shaft 40 in compression to change the functional features of these elements, as described below.

Rotational movement of actuator 80 rotates the rotation control shaft 40, guide arm 82, and actuation catheter 39. Rotation control shaft 40 is a laser-cut hypotube designed to be flexible and to transmit the rotational force along the length of the anchor control catheter. Rotation control shaft 40 is configured to effectively transmit torque from actuator 80 to guide arm 82 to ensure that rotation of the proximal end of rotation control shaft 40 results in a substantially equal amount of rotation of guide arm 82 at the distal end of rotation control shaft 40 (e.g., approximately 1:1 rotation ratio between the control shaft and the guide arm). Each region of rotation control shaft 40 is cut in a pattern (or not cut at all) to provide features useful to that region.

Tether 42 extends from an actuator 90 in the proximal controller to the anchor 88 of the prosthetic heart valve. Tether 42 is releasably attached to anchor 88 at coupling 95 using, e.g., one of the releasable connectors described in WO 2022/046678. Axial movement of tether 42 and anchor 88 is controlled by movement of the actuator 90 in the proximal controller.

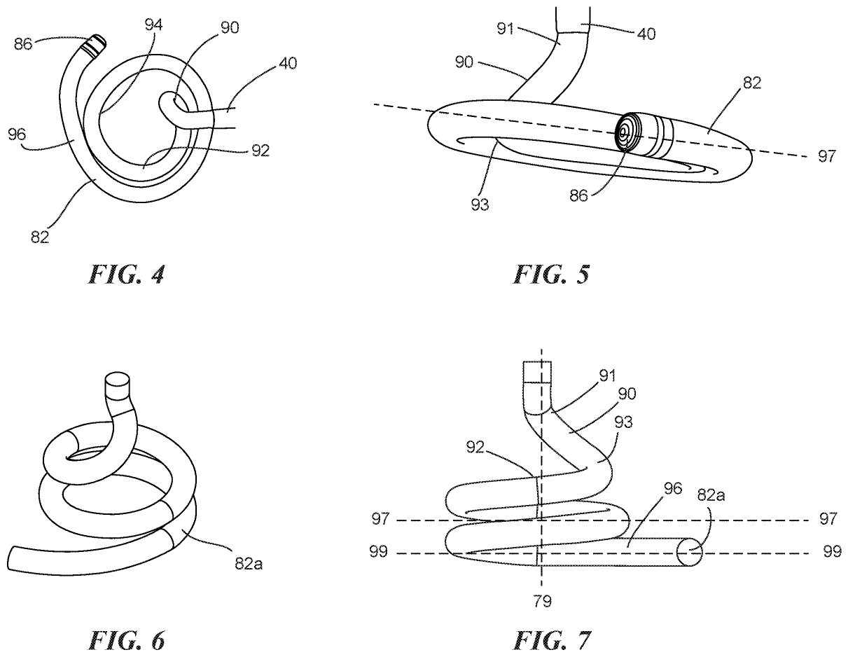

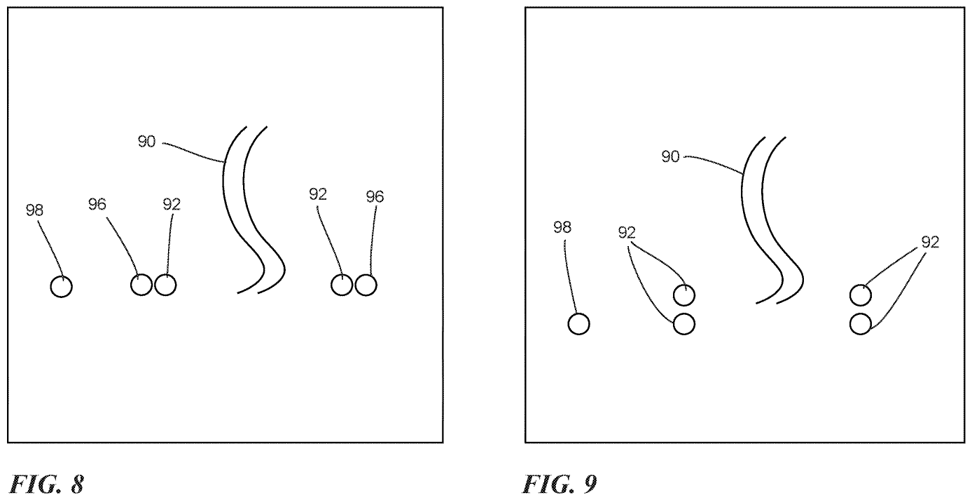

FIGS. 4-7 are embodiments of guide arm 82 and the distal end of rotation control shaft 40. As shown, guide arm 82 is in a spiral configuration (FIGS. 4-5) and/or helical configuration (FIGS. 6-7), such as the configuration it would controllably assume after emerging from the distal end of the inner steerable catheter in the left atrium of the heart. The geometry of the guide arm provides a consistent self-assembly with a proven encircling geometry. As will be described in more detail, the anchor control catheter is used as the encircling tool which decouples the anchor itself from (e.g., direct) encircling.

Referring to FIGS. 4-5, guide arm 82 has three parts: A curved part 90 extending radially outwards from a joint at the distal end of the rotation control shaft 40, an intermediate part 92 extending from the curved part 90 to a transition point 94, and a distal part 96 extending from the transition point 94 to the distal end of the guide arm that includes cap 86. The rotation control shaft 40 generally extends axially along a longitudinal axis of the anchor delivery subsystem and three nested catheters. The curved part 90 of the guide arm 82 bends at bend 91 and extends radially outwards before bending again sharply inwards at bend 93 as it transitions to the intermediate part. The distal part 96 and the intermediate part 92 generally lie in a plane 97 that is orthogonal to axis 79 of the rotation control shaft 40 (or the longitudinal axis of the anchor delivery subsystem). The curved part and bends 91 and 93 facilitate the transition of the guide arm 82 from aligning with the longitudinal axis of the rotation control shaft to the proximal and distal parts laying in the plane that is orthogonal to the longitudinal axis. This embodiment of the guide arm 82 is described as having a spiral configuration.

FIGS. 6-7, shows a view of another embodiment of a guide arm 82a that has a helical configuration in contrast to the spiral configuration of guide arm 82. As with the embodiment above, the guide arm 82a includes a curved part 90 that extends radially outward from the rotation control shaft 40 between bends 91 and 93. However, in this embodiment, the intermediate part 92 and the distal part 96 do not lie together in a single plane that is orthogonal to the longitudinal axis of the anchor delivery subsystem or nested catheters. Instead, in this embodiment, the intermediate part comprises a helical section that includes turns (e.g., loops) axially spaced apart (e.g., that reside in more than one plane). As can be seen, depending on the number of turns, this results in the proximal portion turning in a helical fashion so as to lie in at least planes 97 and 99 before transitioning to distal part 96 which lies in plane 99. In the illustrated example, the intermediate part can include, for example, less than two turns. FIGS. 6-7 show the helical guide arm 82a, including how the distal and intermediate parts in guide arm 82a lie in planes 97 and 99, in contrast to the in-plane arrangement of guide arm 82 (see FIG. 5). The geometry of the guide arm, for example the distal part 96 lying in a plane 99 that is generally orthogonal to axis 79 of the rotation control shaft, facilitates delivery of the anchor into a planar arrangement with the mitral annulus. The anchor delivery system is designed so as to maintain the orthogonal arrangement of the guide arm to the rotation control shaft throughout the delivery procedure, which, along with independent user control of system rotation, grabber arm reach, and system (axial) position, enables repeatable and fine-tuned control of depth/radial extent to the clinician during encircling.

Since the guide arm is not implanted or left behind in the patient, inclusion of visualization features or markers thereon that facilitate imaging in real-time such as with ultrasound and/or fluoroscopy can be made without concern for the impact such features would have on implant (e.g., anchor) delivery or performance. For example, the guide arm can include radiopaque markers to allow for this visualization. Additionally or alternatively, the construction of the anchor control catheter with laser cut shape memory or nitinol tubing provides highly reflective features that are easily visualized via ultrasound.

FIG. 8 is a representation of a sample cross-sectional echo image of the guide arm 82 from FIGS. 4-5. As shown in FIG. 8, the curved part 90 is visible under ultrasound along with circular cross-sections in a single plane representing the intermediate part 92, distal part 96, and distal tip 98 of the guide arm 82. FIG. 9 is a representation of a sample cross-sectional echo image of the guide arm 82a from FIGS. 6-7. In this image, since the intermediate part 92 makes more than one helical turn, the intermediate part rests in more than one plane, so the circular cross-sections of the intermediate part 92 are readily visualized and distinguished in the ultrasound image. Additionally, this allows for easier visualization of the distal tip 98 as it is distinguishable as a single circular cross-section spaced apart from the paired cross-sections of the helical intermediate part. This results in easier visualization of the distal tip of the guide arm 82a, which can help the user to encircle selected anatomy with the distal tip since it is more distinct on echo imaging.

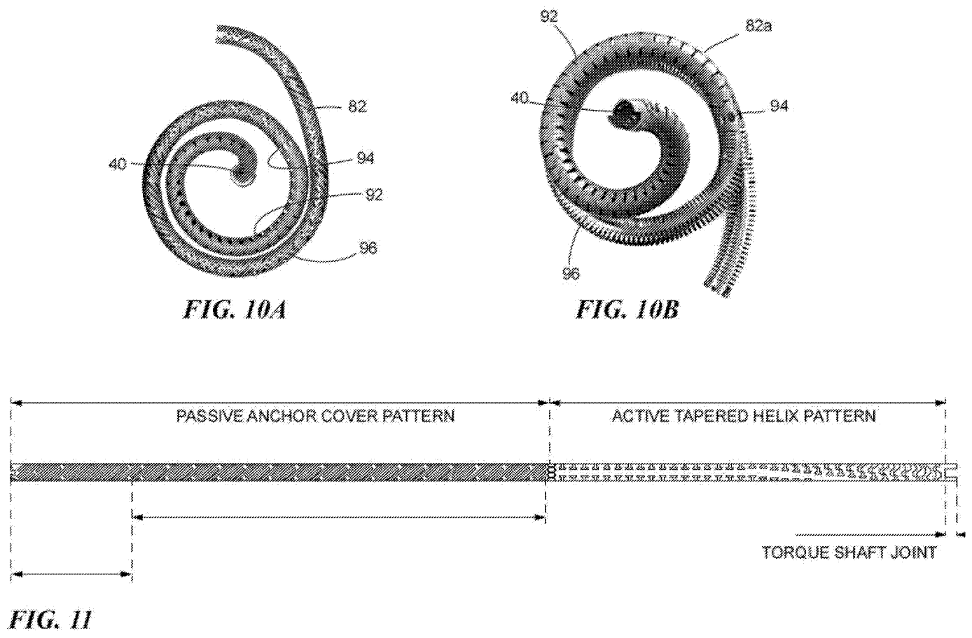

Referring to FIGS. 10A, 10B and 11, the guide arm can be a shape memory material laser cut in a combination of active (e.g., 92) and passive (e.g., 96) sections, with transition region 94 and a tether coupling portion 95. Active section 92 can comprise a tapered helix pattern with a longitudinal spine 102 extending generally helically in a proximal portion thereof, and longitudinally in a distal portion of the active section 92. A series of windows 104 can be disposed opposite to spine 102, and a pair of toothed sections 106 are disposed 90° apart from the windows 104 and spine 102. In some embodiments, passive section 96 comprises a generally spiral cut pattern with periodic bridge structures. In some embodiments passive section 96 comprises a longitudinal spine (or spines) with spaces (or cuts) disposed on radially inward and/or outward aspects of the guide arm (providing radial flexibility and axial stability). This combination of these active and passive sections, and the preset (shape memory) shape, of cause the guide arm to assume a spiral (e.g., 82, FIG. 10A) or helical (e.g., 82a, FIG. 10B) shape (depending on if the configuration of FIGS. 4-5 is used or the configuration of FIGS. 6-7) when it emerges from the steerable catheter. Proximal movement of actuation catheter 39 with respect to guide arm 82 (FIG. 3) engages opposed edges of the tapered helix cut pattern to lock the active section 92 into the desired shape, i.e., extending from the longitudinal axis of the inner steerable catheter through an approximately 90 degree bend to the flat spiral or helical shape described above. Since the shape of the anchor control catheter is formed by a shape memory or nitinol laser cut tube, the complex surface features reflect acoustically quite well and enable distinctive echo visualization.

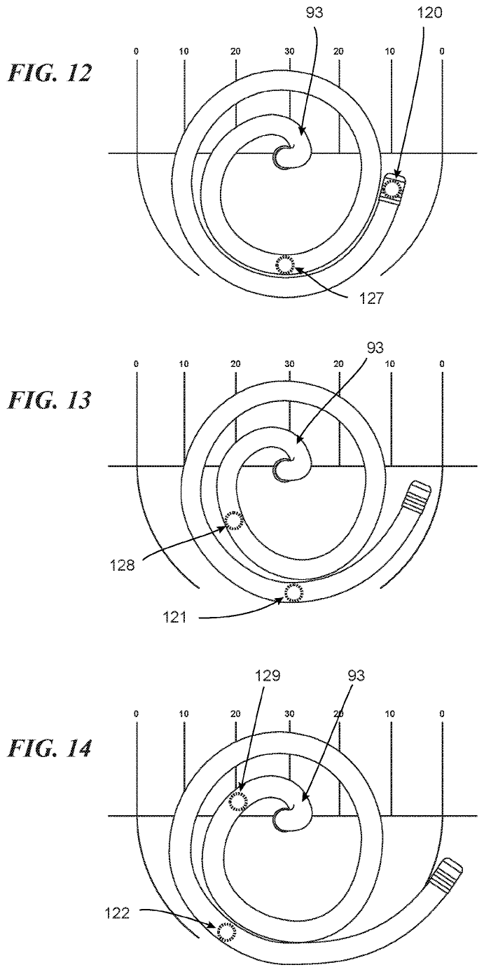

The distal part 96 is also configured to be manipulated from its set shape into more open and/or more closed shapes by moving tether 42 to provide proximal and distal movement of anchor 88 within distal part 96 of guide arm 82 (FIG. 3). The support structure of distal part 96 can be laser cut in a pattern of alternating spiral components 98 and bridges 100 that provide flexibility so that proximal and distal movement of the stiffer anchor within intermediate part 92 and distal part 96 can bend or straighten distal part 96. The shape of the distal part 96 can also be controlled by movement of actuation catheter 39. FIG. 12 shows the guide arm in a self-assembly position in which the distal portion of the anchor within the guide arm is deployed to a depth indicated by arrow 120 and the proximal portion of the anchor within the guide arm is deployed to a depth indicated by arrow 127. In the self-assembly position, the shape and depth of the anchor within the guide arm results in the distal tip and distal part of the guide arm resting against itself as shown. Such a configuration provides a minimized “envelope” of the guide arm, assisting in 1.) deployment of the guide arm within the atrium, and 2.) advancement of the anchor control catheter from the atrium to the ventricle—without deleterious contact/entanglement with native tissue. FIGS. 13-14 show the guide arm in an encircling position in which the distal portion of the anchor is retracted within the guide arm to a depth indicated by arrows 121 and 122, and the proximal portion of the anchor is retracted within the guide arm to a depth indicated by arrows 128 and 129, respectively. As shown, retracting the anchor results in the distal tip and distal part of the guide arm extending outwards, while maintaining the proximal portion of the anchor distal to bend 93 of the guide arm. This allows for fine user control of the angle and distance of the distal tip from the rest of the guide arm to assist the user with encircling of the chordae. Keeping the proximal portion of the anchor distal to the bend 93 of the guide arm maintains the prescribed geometry of the guide arm while enabling adjustability of the distal tip of the guide arm during encircling.

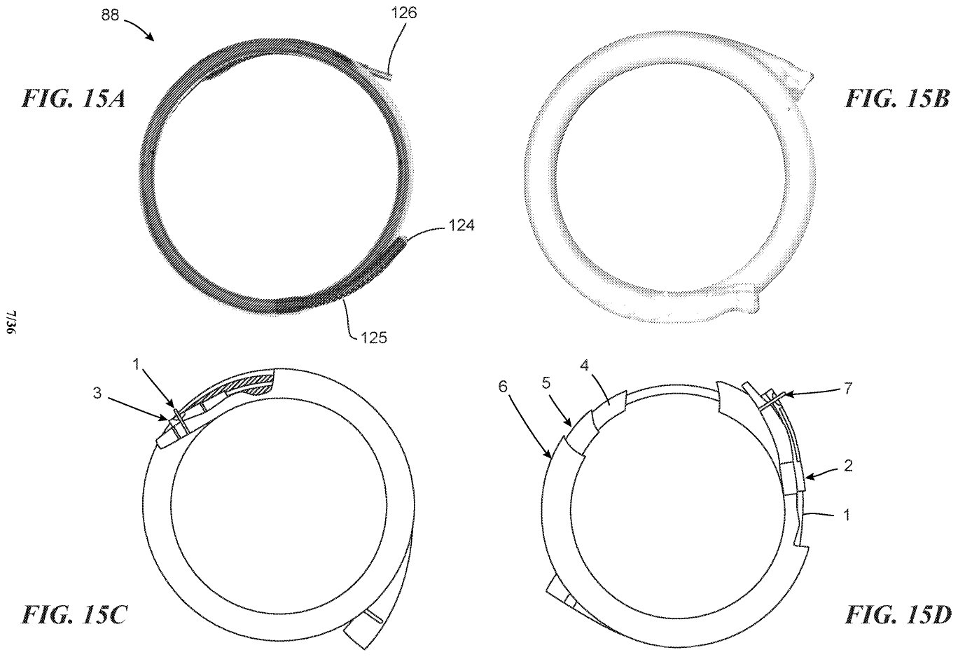

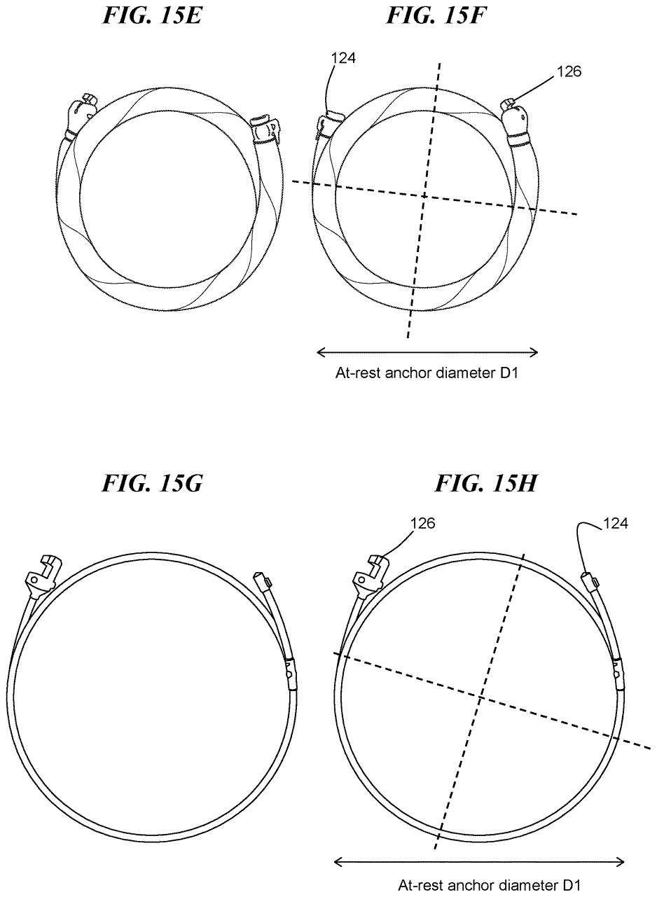

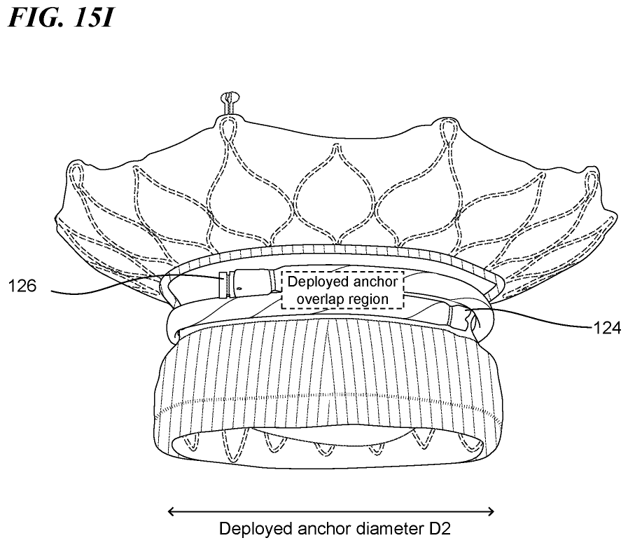

FIGS. 15A-15D show details of anchor 88. When loaded into the anchor control catheter, anchor 88 assumes a generally straightened shape. In its unconstrained state, anchor 88 extends in a spiral from a distal tip 124 to a proximal connector 126, where it releasably attaches to the tether. A laser-cut segment 125 at the distal end is more flexible than a central portion of the anchor and is therefore less traumatic to the delivery system and the patient's anatomy. A flexible laser-cut segment may be provided on the proximal end of the anchor as well. Using the inner steerable catheter 36 for steering under ultrasound and/or fluoroscopic visualization, the spiral portion of the anchor control catheter 38 is then advanced through the leaflets of the native valve.

FIG. 15B shows the anchor of FIG. 15A including one or more layers of expanded polytetrafluoroethylene (ePTFE) disposed over the anchor to provide a lubricious and biologically inert coating that protects the anatomy from damage or abrasions that could otherwise be caused by uncoated metal. While ePTFE is used in this embodiment, it should be understood that other similar materials can be used with the anchor. FIGS. 15C-15D provide additional cutaway views of the anchor, including a core 1 (e.g., a shapeset material such as nitinol), a distal tip 2, and a proximal termination assembly 3. The anchor can include one or more ePTFE coatings, in this embodiment the anchor includes inner coating 4, middle coating 5, and outer coating 6. Furthermore, the anchor can include suture points 7 on one or both of the proximal and distal tips that can be used to secure the one or more coatings to the anchor.

FIGS. 15E-15F show examples of anchors with one or more layers of ePTFE disposed over the anchor, and FIGS. 15G-15H show examples of anchors without the layers of ePTFE (e.g., the bare metal portion of the anchor). In FIGS. 15E-15F, suture lines are shown on the anchor which attach the one or more layers of ePTFE to the underlying anchor. FIGS. 15E-15H are intended to show an at-rest shape of the anchor, including the number of turns of an exemplary anchor (i.e., the amount of (e.g., helical) overlap of the anchor between the proximal connector 126 and the distal tip 124. For example, referring to FIGS. 15F and 15H, it can be seen that the anchor can have an at-rest shape comprising approximately 1.5 turns, approximately 1.6 turns, approximately 1.7 turns, or approximately 1.8 turns. In some embodiments, the number of turns or overlap of the anchor can be defined as the amount of degrees of rotation of the anchor. For example, the anchor may comprise a (e.g., helical) anchor spanning up to 540 degrees, up to 585 degrees, up to 630 degrees, or up to 675 degrees. As described above, the number of turns, or degrees of rotation of the anchor, can refer to the length of anchor without the distal tip 124 or proximal connector 126.

As is also shown in FIGS. 15F and 15H, the anchor can have an at-rest diameter D1. This at rest diameter can be the diameter of the anchor when it assumes its shape-set shape (e.g., after deployment from a delivery device).

FIG. 15I shows an anchor with a heart valve replacement frame self-expanded within the anchor. This represents the configuration when the anchor and valve frame are delivered into the heart of a subject. It should be understood that when the anchor is delivered or deployed, forces acting upon the anchor can change the diameter of the anchor. For example, forces acting on the anchor from chordae in the heart, or from a heart valve replacement frame, may cause the anchor to expand to a deployed diameter D2 that is larger than the at-rest diameter D1. In some aspects, the delivery or deployed diameter D2 of the anchor may be up to 20% larger, up to 25% larger, up to 30% larger, or up to 35% larger than the at-rest diameter D1. In one specific example, the deployed diameter D2 is 20-25% larger than the at-rest diameter D1. In one specific example, an at-rest diameter of the anchor may comprise approximately 23 mm, and a deployed or delivery diameter of the anchor may comprise approximately 28 mm (e.g., when the valve frame is self-expanded within the anchor).

The self-expanding heart valve frame and free-ended anchor, when implanted, together form a system that reaches an equilibrium with respect to size and relative position of the two components. Equilibrium is reached when the force from the expanding heart valve, which is returning from a collapsed (e.g., crimped) state, equals the reactive force of the (e.g., at rest) anchor. The stiffness of the at rest anchor is insufficient to fully constrain the valve frame that is expanding therein, but increases-for example, via spring force-as the anchor is further expanded from its rest state. This causes the anchor to increase in diameter until a balance of forces between the valve frame and anchor is achieved. The initial low interaction forces between the (e.g., expanding) valve and anchor in or near its at rest state enable relative axial movement. For a heart valve frame having a reduced diameter within a central portion (e.g., waist, or landing zone), the interaction forces between the expanding heart valve frame and anchor will tend to direct the anchor toward the smaller diameter. When a heart valve frame is self-expanded within the anchor, in addition to the diameter of the anchor increasing, the number of turns or radial length/overlap of the anchor to change (e.g., be reduced). This is enabled at least in part by the free-ended construction of the anchor. For example, self-expansion of the heart valve replacement frame into the anchor is configured to reduce a number of turns of the helical anchoring device. In some examples, a helical anchoring device comprising approximately 1.6 to 1.8 turns in the at-rest configuration and the number of turns is reduced to between 1.1 to 1.25 turns in the deployed configuration when the valve frame is self-expanded within the anchor. Similarly, the amount of rotation of the anchor may be reduce from between 630 to 675 degrees to between 540 to 585 degrees of rotation.

To deliver an anchor to a patient's heart, the outer steerable catheter 34 and inner steerable catheter 36 are used to navigate the delivery system within a sheath (not shown) to the patient's right atrium RA and through the septum to the left atrium LA. The anchor control catheter includes flexibility and smooth rotational control of the catheter across the septum. The guide arm 82 of the anchor control catheter 38 is then advanced out of the distal end of the inner steerable catheter 36 where it assumes a spiral shape or helical shape under the control of the shape set of the intermediate part of control arm 82 and the control of the distal part of control arm 82 by actuation catheter 39. The anchor also fully self-assembles and assumes it's at-rest shape within the guide arm of the anchor control catheter within the left atrium. When positioned with its distal end generally adjacent to the distal end of the anchor control catheter, the smaller radius or profile of the anchor with respect to the distal arm radius drives a smaller self-assembly envelope of the anchor control catheter within the left atrium.

Using the inner steerable catheter 36 for steering under fluoroscopic visualization, the spiral or helical portion of the anchor control catheter 38 is then advanced through the leaflets of the native valve 130 into the left ventricle LV. Using the actuation catheter 39 to extend the distal tip of guide arm 82 radially outwards from the guide arm, the anchor control catheter 38 is rotated within the left ventricle to advance guide arm 82 between the chordae and the heart wall with the anchor still inside the anchor control catheter. Because the anchor is stiffer than the guide arm, as described above, the position of the anchor with respect to the distal part of the guide arm can also be used to conform that portion of the guide arm to the spiral shape of the anchor (e.g., the radius of the anchor). The chordae can then be encircled with the guide arm for at least the full length of the anchor (e.g., approximately 1.5 full turns). Once the anchor and guide arm are in place, one procedural strategy to reduce left ventricular outflow tract obstruction (LVOTO) from the prosthetic valve is to implant the anchor as high as possible within the anatomy. Satisfactory encircling of chordae and/or leaflets can also be assessed via ultrasound and/or fluoroscopy, on account of the echogenic and radiopaque features of the anchor control catheter and/or the anchor. This can be accomplished by lifting up on the anchor prior to pulling the anchor towards the left atrium. In some embodiments, this adjustment is made by pulling on the anchor control catheter, since the anchor still resides within the guide arm. In other embodiments, if the anchor has already been deployed from the guide arm, the anchor can be repositioned or pulled up or towards the atrium with the tether.

To fully deploy the anchor in the left ventricle, the anchor control catheter 38 is withdrawn from the anchor while holding tether 42 stationary. The anchor control catheter 38 is withdrawn into the inner steerable catheter 36 until the distal end clears the proximal end of the anchor. The tether is then decoupled from the anchor, the inner steerable catheter 36 is withdrawn into the outer steerable catheter 34, and the anchor delivery subsystem is withdrawn from the patient. The anchor does not lose chordae and remains stable during anchor control catheter retraction. The anchor control catheter retraction is a simple process.

The anchor delivery subsystem including the anchor control catheter and guide arm described above provides a purpose-built chordal encircling tool configured to deliver the anchor. As described above, the anchor delivery subsystem is configured to safely assemble the anchor within the anchor control catheter in the left atrium and away from the chordal apparatus. The anchor delivery subsystem includes a small envelope with low tip penetration power to avoid contact with the left atrium. The configuration provides a simplified system with only a single tube (e.g., the anchor control catheter guide arm) encompassing the anchor. When encircling within the left ventricle, only the fully self-assembled anchor control catheter touches the chordae, and there is little to no friction stack-up during encircling as can occur in alternative devices.

When the anchor control catheter and anchor are advanced from the left atrium into the left ventricle, the anchor control catheter provides excellent visualization in a single echo plane with standard echocardiographic views in the left ventricle during encircling. As described above, a user can easily visualize the turns of the anchor control catheter with distinct visualization of the intermediate portions and of the tip of the guide arm. Encircling control is enabled in both depth (e.g., inferior/superior) control-by advancing/retracting the anchor control catheter, and independently, reach (e.g., radial) control-- by axial movement of the anchor within the anchor control catheter to modify its radial reach at the distal tip thereof. The combination provides a user with fine-tuned adjustability of encircling position within the heart. This visualization combined with control of the tip greatly simplifies the encircling process to navigate a variety of patient anatomies.

Additionally, the encircling process is easily reversable as many times as required to capture the intended chordae and get the anchor in position. If chordae are missed or the user is not satisfied with the position of the anchor control catheter or anchor, both can be repositioned simply by unwinding the encircling and starting the process again. The simplified design of the anchor control catheter and limited cross-sectional diameter further provide stable hemodynamics throughout the anchor delivery process.

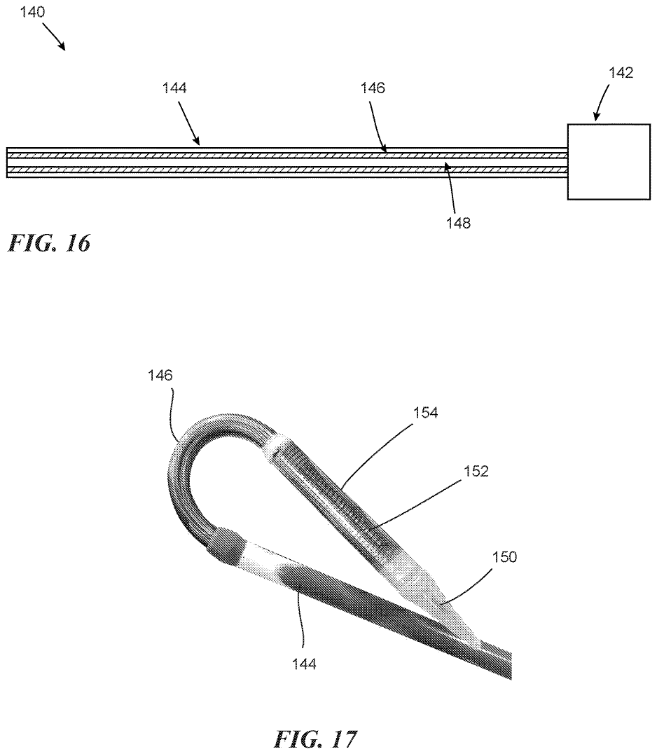

FIG. 16 shows aspects of a valve delivery subsystem 140 having a proximal controller 142 and three nested catheters: An outer steerable catheter 144, a capsule shaft 146 movably disposed in the lumen of the outer steerable catheter 144, and an inner steerable catheter 148 movably disposed in the lumen of the capsule shaft.

FIG. 17 shows a view of the distal end of the valve delivery subsystem, including outer steerable catheter 144, capsule shaft 146, nose cone shaft 150, and valve capsule 152. A guidewire (not shown) may be disposed in a lumen of nose cone shaft 150. The outer steerable catheter and inner steerable catheter may be configured like the outer steerable catheter and inner steerable catheter described above with respect to the anchor delivery subsystem. As shown in FIG. 17, capsule shaft 146 is shown extending distally of the distal end of outer steerable catheter 144. The valve capsule 152 is configured to contain a compressed prosthetic valve 154 attached to the distal end of the capsule shaft 146.

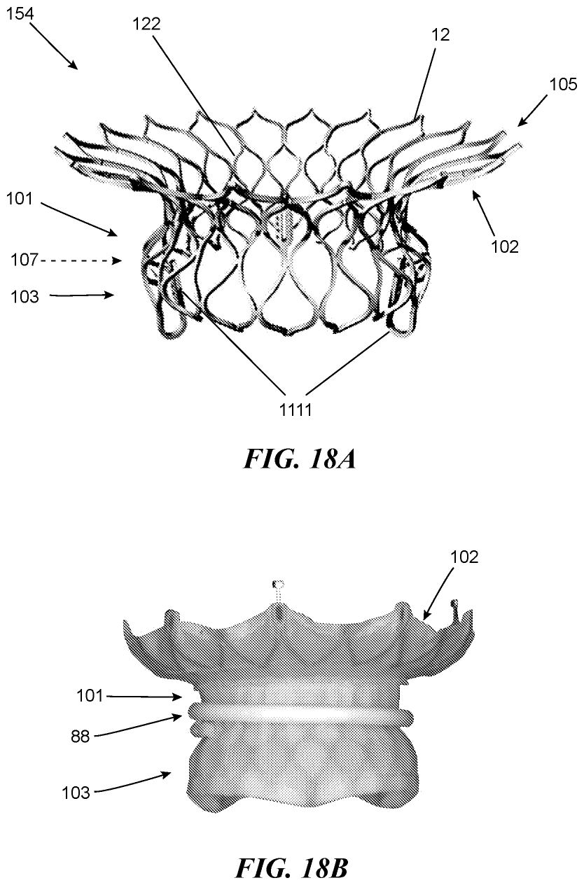

FIGS. 18A-18B show a valve prosthesis 154 having a valve frame structure 12 and being configured to support a plurality of leaflets (not shown) therein. As described above, the valve prosthesis can be delivered into the deployed anchor with the valve delivery subsystem described above. The valve frame 12 can include interior commissure attachment mechanisms 1111 for attachment of the leaflets to the frame structure 12. The valve frame structure 12 can be deployed from a collapsed (delivery) configuration via the valve delivery subsystem to an expanded configuration during a procedure for replacing or repairing a native valve, such as a mitral valve. The valve frame 12 can include a plurality of rows (e.g., 3-7 rows) of substantially diamond-shaped cells 122. The valve frame structure 12 can be configured to foreshorten during delivery (i.e., as the valve frame structure 12 transitions from the collapsed configuration to the expanded configuration) due to the cell structure. In some embodiments, the valve frame structure 12 can be configured to self-expand from the collapsed configuration to the expanded configuration (e.g., can be made of a shape memory material such as nitinol). The valve frame 12 can provide circumferential strength and/or longitudinal strength to valve prosthesis 154.

The valve prosthesis 154 can be deployed in an expanded configuration according to the methods described herein. In the expanded configuration, valve prosthesis 154 can be positioned and/or anchored at a target region of a subject (e.g., an organ or tissue of an animal such as a dog, cat, horse, or human). For example, valve prosthesis 154 can be positioned in the expanded configuration in the orifice of a heart valve, such as the mitral valve or tricuspid valve (e.g., to function as a temporary or permanent replacement for an existing mitral valve or tricuspid valve of the heart).

One or more portions of the valve frame structure 12 can be shaped or configured to aid in securing the valve frame structure 12 at a location (e.g., in the orifice of a native heart valve). For example, the valve frame structure 12 can include an atrial flared portion 102 and a ventricular portion 103 configured to help secure the frame in the anatomy. The atrial flared portion 102 and ventricular portions 103 can extend radially outwards from a narrow central waist portion 101. The atrial flared portion 102 can, for example, be configured to extend into the atrium of the heart from the central waist portion 101 when the valve prosthesis is deployed in the native mitral valve. The ventricular portion 103, in turn, can extend into the ventricle of the heart from the central waist portion 101 when the valve prosthesis is deployed in the native mitral valve. The narrow central waist portion 101 is configured to engage with the anchor previously described. The atrial flared portion 102 and ventricular portion 103 can, for example, be configured to be positioned on either side of the anchor 88 (e.g., that is wrapped around the chordae and the central waist portion) to anchor the valve frame structure 12 in the anatomy. Alternatively or additionally, the atrial flared portion 102 and ventricular portions 103 can be configured to engage with tissue to prevent the valve prosthesis from slipping through the native valve orifice.

Referring to FIG. 18A, the frame structure 12 of the valve prosthesis 154 can be in a partial hourglass shape such that the ventricular portion 103 initially flares radially outwards from the central waist portion 101 in the ventricular direction (e.g., above 107), but then curves back inwards towards a center of the frame structure (e.g., below 107). As shown, the interior commissure attachment mechanisms 1111 can be positioned on an interior of the ventricular end of the ventricular portion 103. In some embodiments, the interior surface of the interior commissure attachment mechanisms 1111 can be configured to align radially with the narrow central waist portion 101. This half-hourglass or cup shape of the ventricular portion 103 can advantageously help provide space for the chordae therearound. The ventricular portion 103 is designed to be as short as possible (e.g., having a length of approximately 8 mm to 15 mm in the axial direction) while still achieving the purpose of supporting the attachments to the leaflets and avoiding the commissures/chordae. Additionally, the axial length (or shortness) of the ventricular portion specifically designed to avoid or prevent left ventricular outflow tract obstruction (LVOTO).

As shown in FIGS. 18A-18B, the flared atrial portion 102 also flares radially outwards from the central waist portion in the atrial direction terminating at the wide atrial brim 105. As shown, the atrial brim 105 of the flared atrial portion 102 may curve slightly inwards from the rest of the atrial flared portion, but still points radially outwards from the frame structure 12. The flared atrial portion 102 including the atrial brim is the widest portion of the frame structure, extending out radially further than the ventricular portion. In general, as shown in FIGS. 18A-18B, the atrial flared portion 102 can extend further radially outwards than the ventricular portion 103. Having a larger atrial flared portion can help prevent para-valvular leakage (PVL). The atrial brim 105 can be extremely conformable and compliant to rest against the anatomy without damaging the tissue while also providing sealing without requiring a PVL guard or other additional structure for sealing. Additionally, the size and conformability of the flared atrial portion and atrial brim allows the valve frame structure to be used across large range of anatomies and conditions.

The specific design and shape of the frame structure 12, including the flared atrial portion 102 and the central waist portion 101, and the interaction between the frame structure 12 and the anchor 88, acts to properly seat the frame structure in the atrium. Specifically, when the anchor is placed in the target anatomy (e.g., in the left ventricle, encircling chordae, and positioned “high” near the annulus), engagement between the central waist portion 101 and the anchor 88 acts to pull the frame structure 12, and particularly the flared atrial portion 102 and wide atrial brim 105 “down” toward the native valve to seat the prosthetic valve and form a seal.

The stiffness and compliance of the atrial brim is optimized to assist in expansion of the overall frame structure into the anchor. The flared atrial portion and particularly the atrial brim needs to be sufficiently stiff to tolerate (e.g., initially) non-ideal anchor placement and still achieve full valve expansion. Non-ideal anchor placement can include the anchor being positioned i) at an axial position along the frame other than at the waist, and/or ii) at an angle with respect to the valve frame. The strut or cell patterns of the flared atrial portion have been designed and configured to increase stiffness of the atrial brim to overcome these positioning cases while still allowing the atrial brim to be compliant enough to conform to the anatomy in an atraumatic manner.

In some embodiments, the atrial flared portion 102 is the softest, most compliant, most conformable, or least stiff portion of the valve prosthesis while still having the stiffness required to assume the fully self-expanded configuration when placed within the anchor.

This flexibility allows the atrial portion to conform to the atrium of the patient. The central waist portion and the ventricular portion can optionally be stiffer than the atrial portion. The stiffness of the central annular portion can aid in its self-expansion to the target diameter and engagement with the anchor. In some embodiments, the central waist portion needs to be able to expand against the counterforce of the anchor. In certain embodiments, the stiffness of the anchor is selected such that, upon valve expansion, the anchor is partly expanded by the valve. For example, the anchor expansion can increase a circumference of the anchor such that a number of turns in the as-delivered state of the anchor is reduced by from about 5% to about 25%. For example, an anchor initially having 1.5 turns, and having approximately 25% of reduction in turns upon valve expansion therein, will following implantation retain about 1.13 turns. It will be appreciated that the number of turns can be reduced by any percent within the aforementioned reduction range. In another aspect, the number of turns of the anchor can be reduced from about 1.75 turns to about 1.5 turns or about 1.2 turns, or from about 1.5 turns to about 1.3 turns or 1.1 turns.

The replacement mitral valve of the present disclosure is purpose built for the mitral position. As described above, the atrial brim is wide and compliant so as to seal against PVL in a variety of patient anatomies without injuring the atrial tissue. The replacement valve prosthesis additionally has a short (e.g., less than 10 mm) ventricular brim to avoid LVOT obstruction in a variety of patient anatomies. Implantation of the valve as described above does not block the native valve, so the system provides good hemodynamics without the need for pacing. The valve frame structure of the present disclosure does not block the native valve until the atrial brim deployed, at which point the valve is competent.

Referring back to FIGS. 16-17, prior to delivering the expandable valve prosthesis to the implanted anchor, a guidewire is inserted through the anchor. One technique for placing the guidewire is to advance an inflated balloon catheter through the implanted anchor toward the apex of the heart. The position of the balloon may be monitored with ultrasound. Using a balloon with a large-enough diameter (e.g., >12 mm) helps ensure that the balloon will not pass between groups of chords. Once the balloon has passed through the anchor and into the ventricle, the guidewire can be advanced through the balloon catheter lumen. The balloon catheter is then withdrawn, leaving the guidewire in place for use in advancement of the valve delivery catheters.

The valve delivery subsystem is placed into the patient's vasculature through the same femoral vein introducer sheath used for the anchor delivery and implantation. During navigation of the prosthetic valve through the vasculature, the distal end of the valve delivery subsystem is steered by bending the distal ends of inner and outer steering catheters 148 and 144 as described above with respect to the inner and outer steering catheters of the anchor delivery subsystem. The valve capsule 152 and nose cone 150 are just distal to the distal end of the outer steerable catheter 144 during advancement into the patient's heart.

When the nose cone 150, valve capsule 152, and the distal end of the outer steerable catheter 144 have passed through the septum into the left atrium of the heart, the inner steerable catheter 148 and valve capsule 152 are advanced out of the outer steerable catheter. Once in position, the capsule shaft 146 may be retracted to retract capsule 152 and expose the distal end of valve 154, thereby allowing it to begin to self-expand within the anchor. The partially self-expanded valve may be pulled proximally against the anchor to move the valve and anchor closer to the ventricular side of the native valve annulus.

Thereafter, the capsule shaft is retracted further to expose the proximal end of valve 154 to allow it to fully self-expand. The valve delivery subsystem may then be removed from the patient.

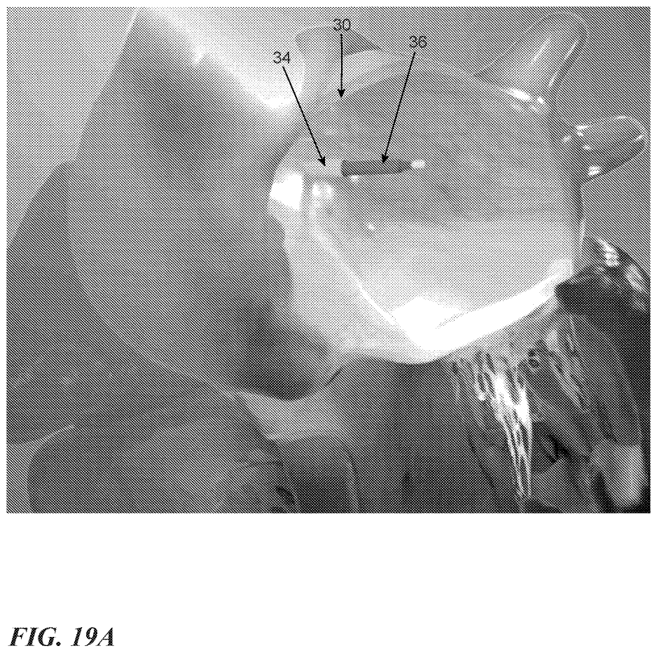

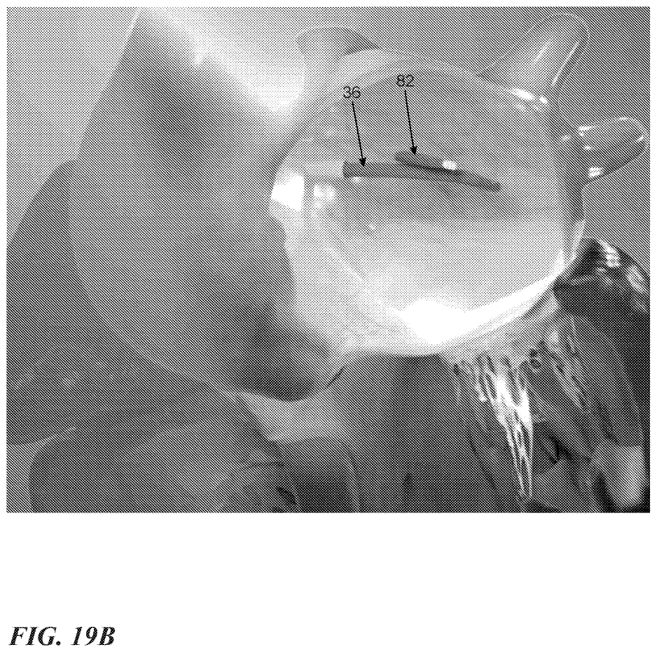

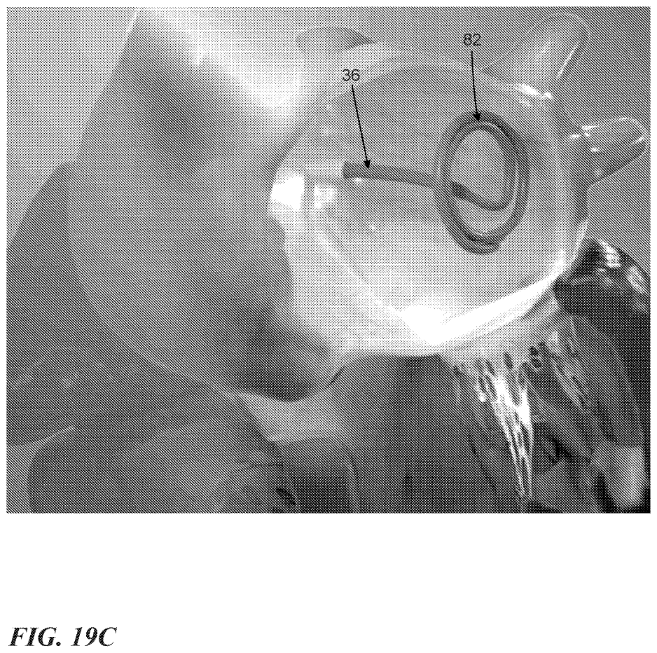

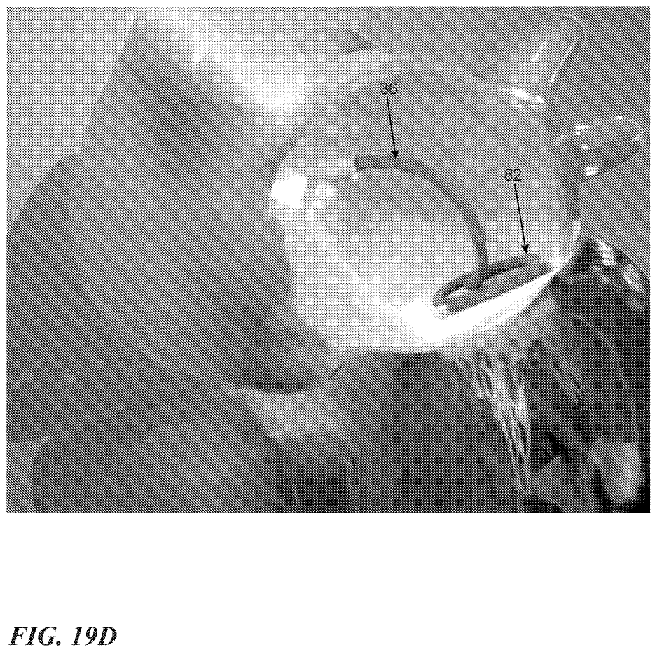

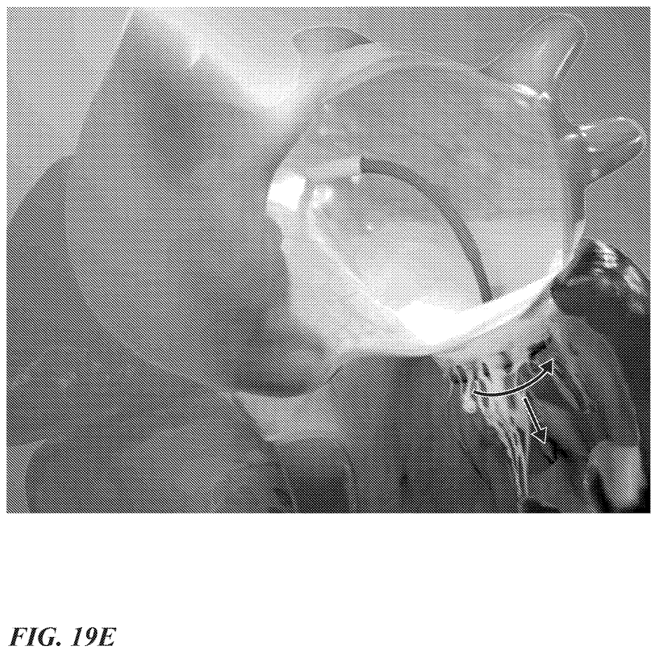

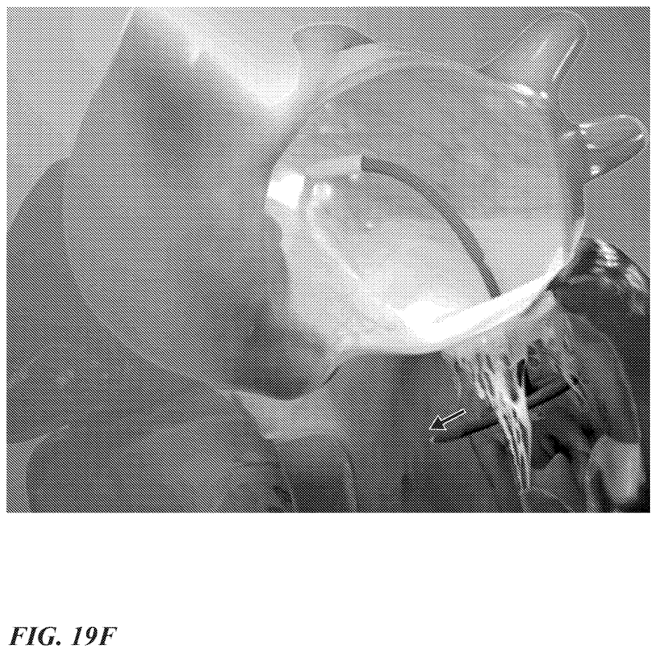

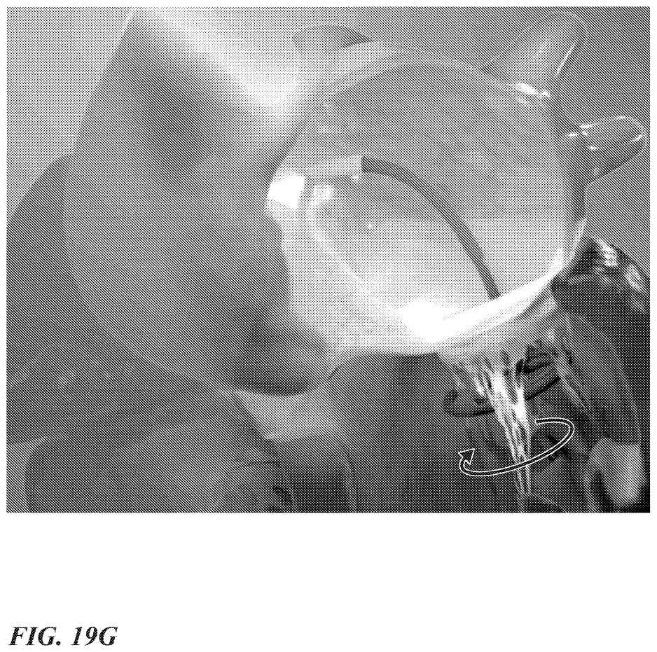

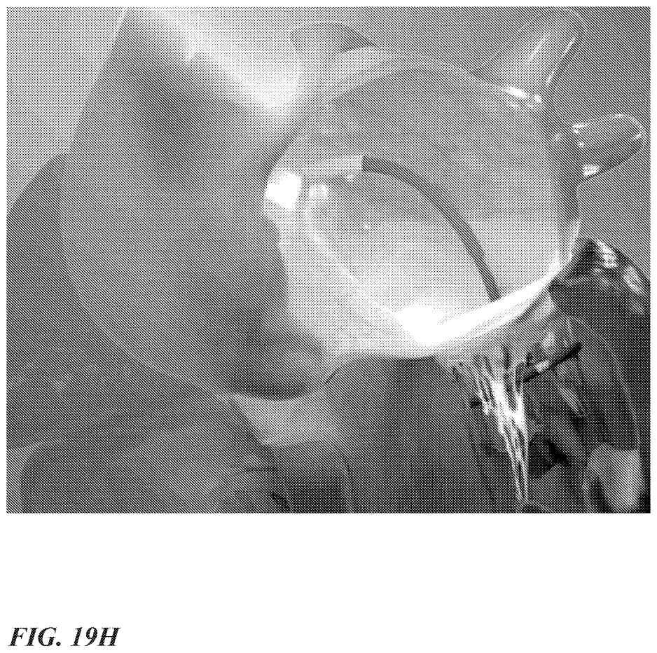

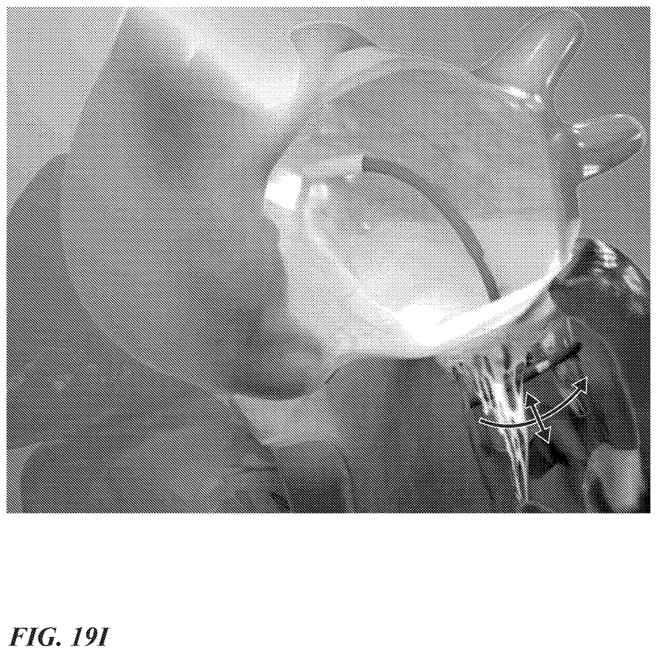

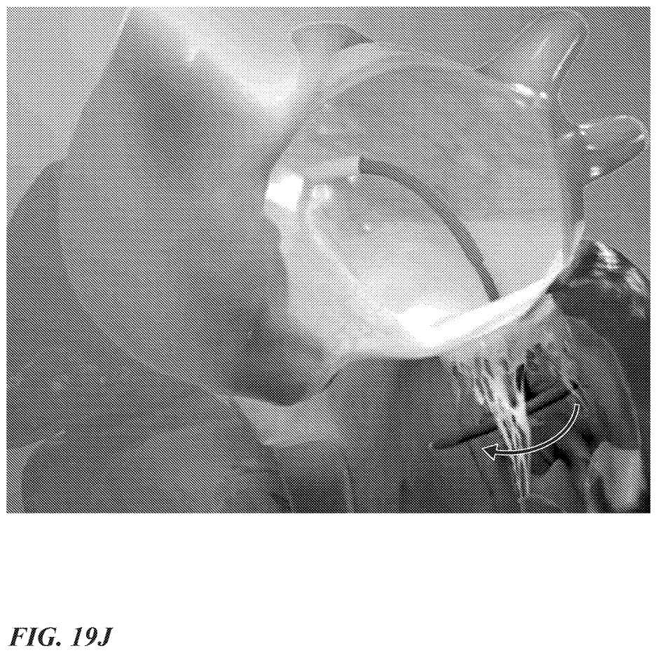

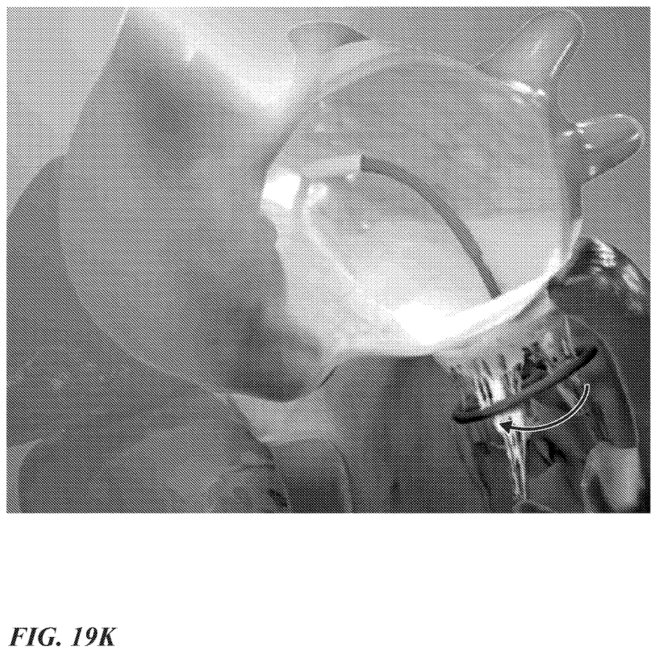

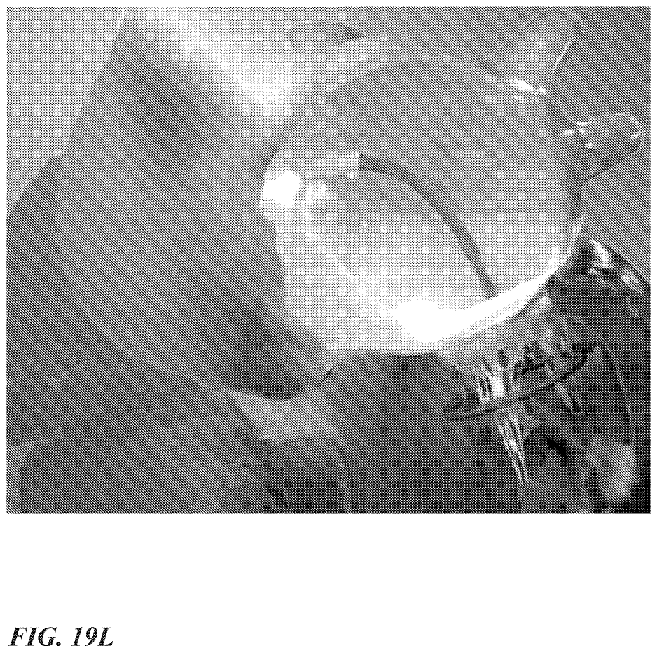

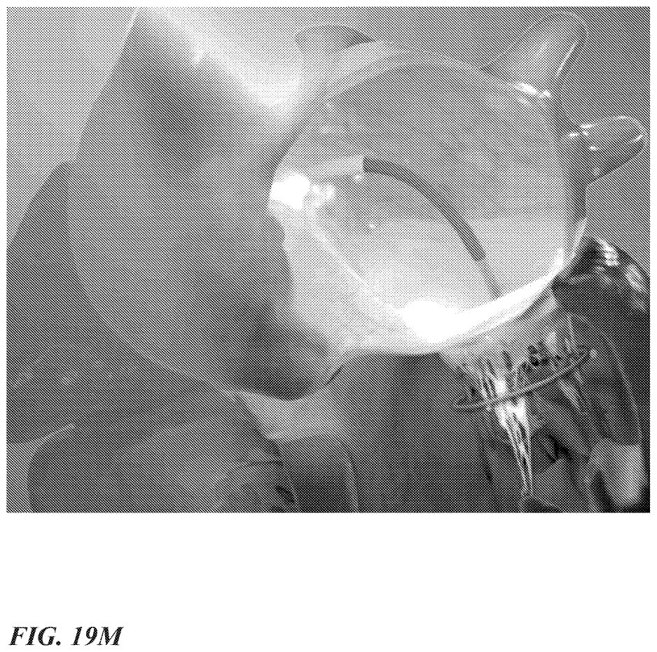

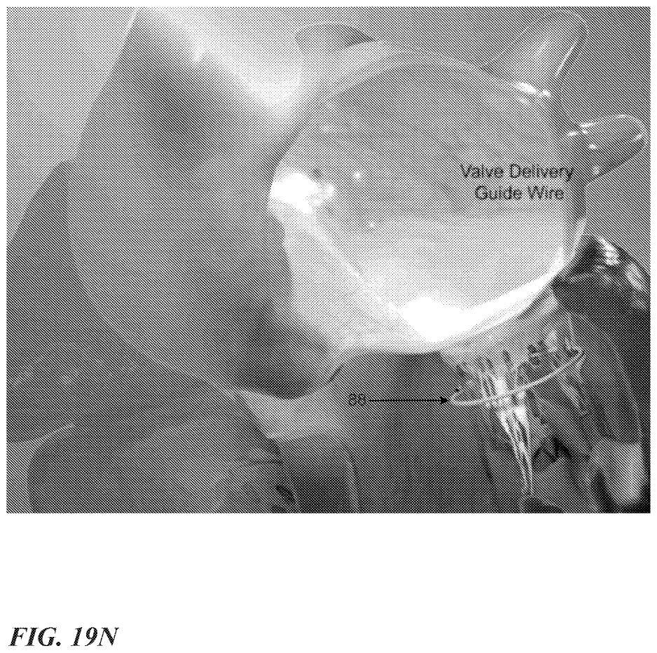

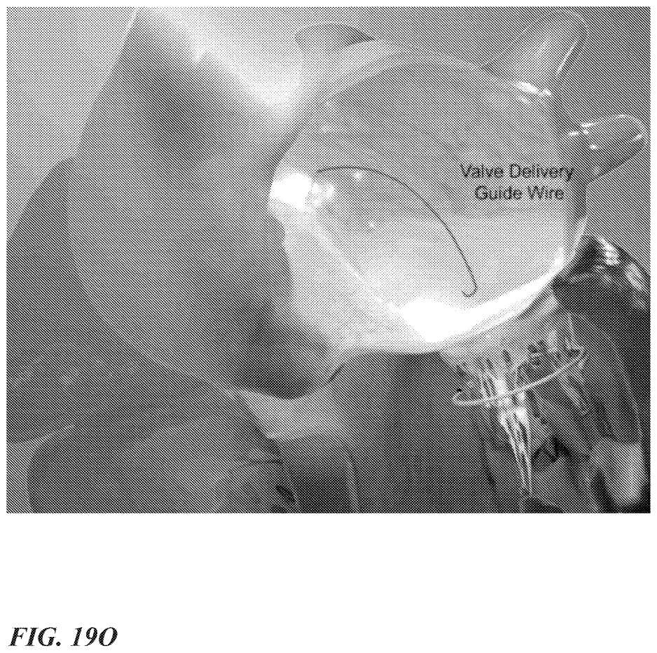

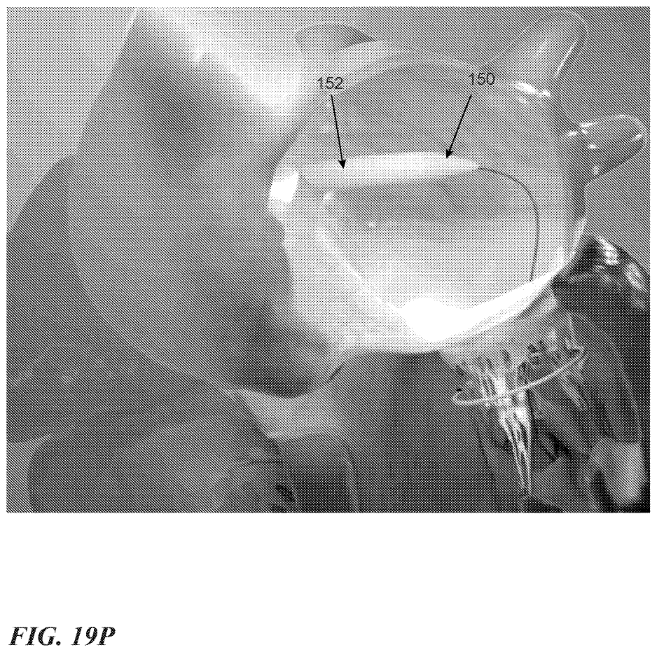

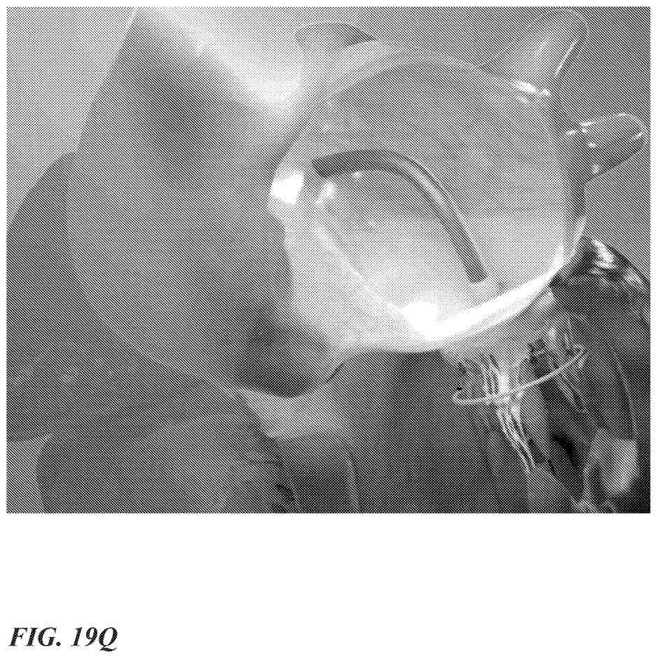

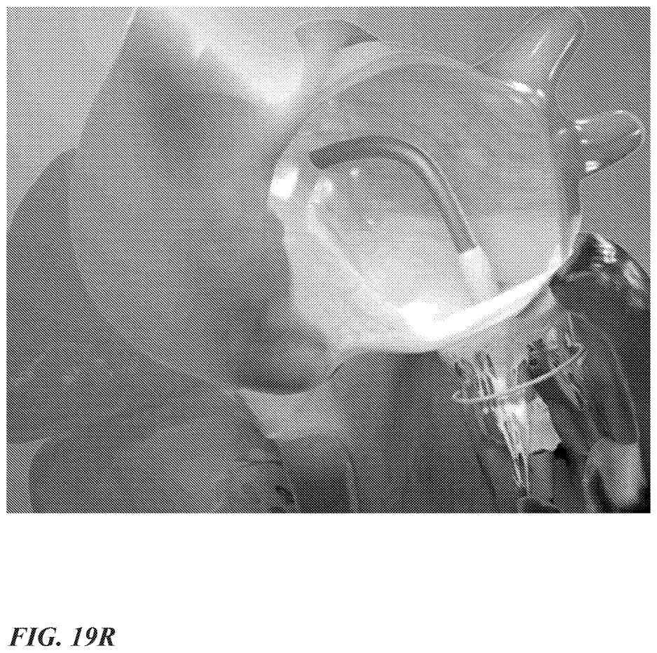

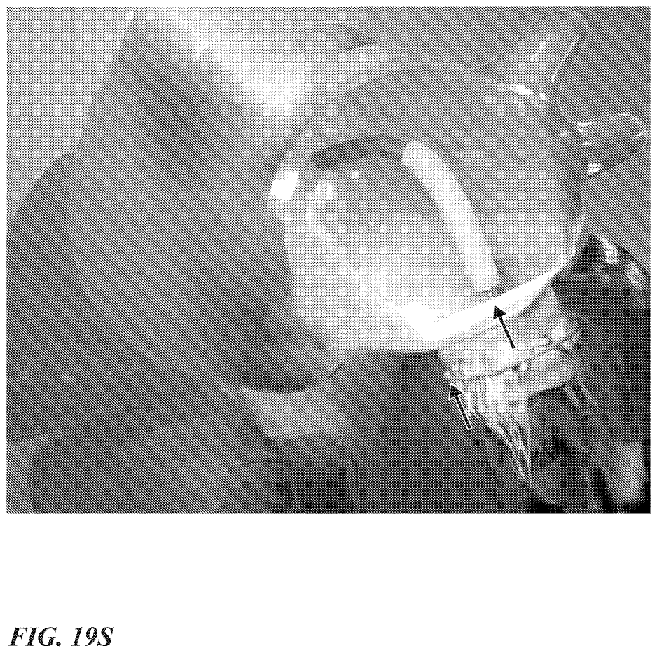

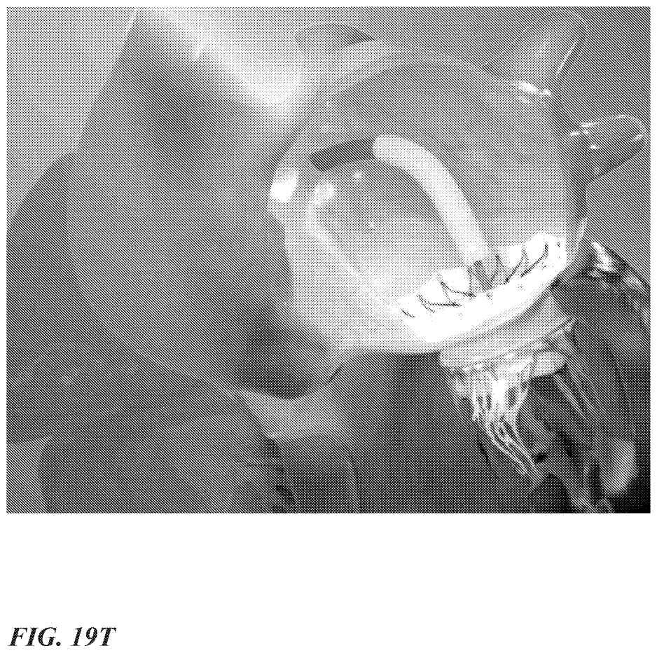

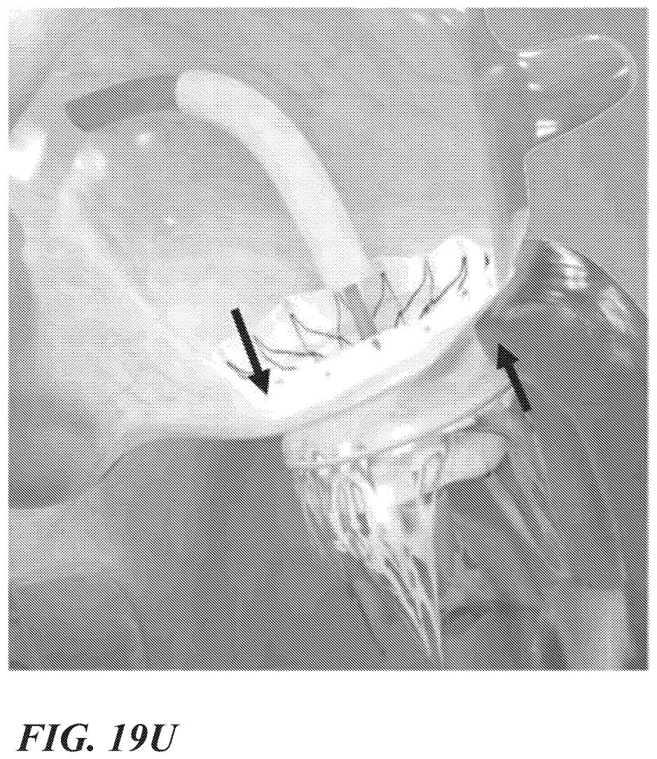

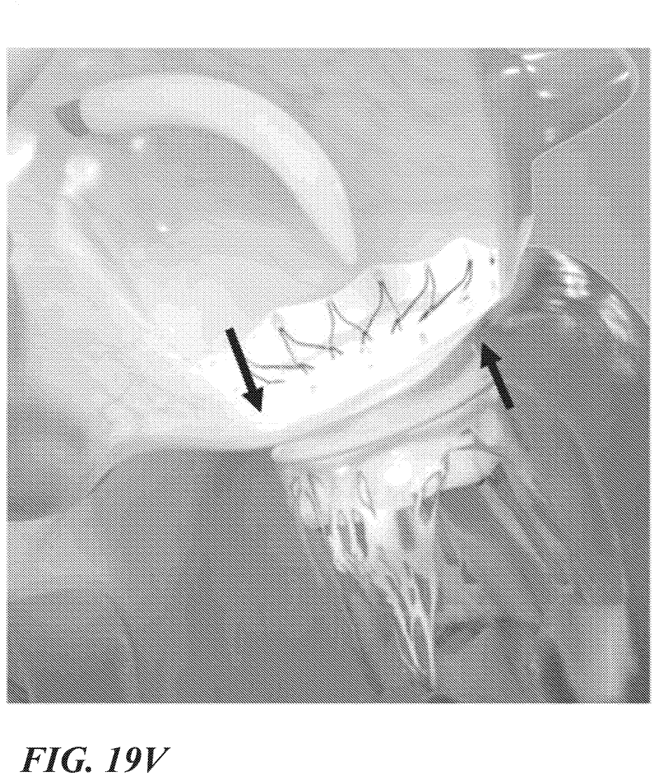

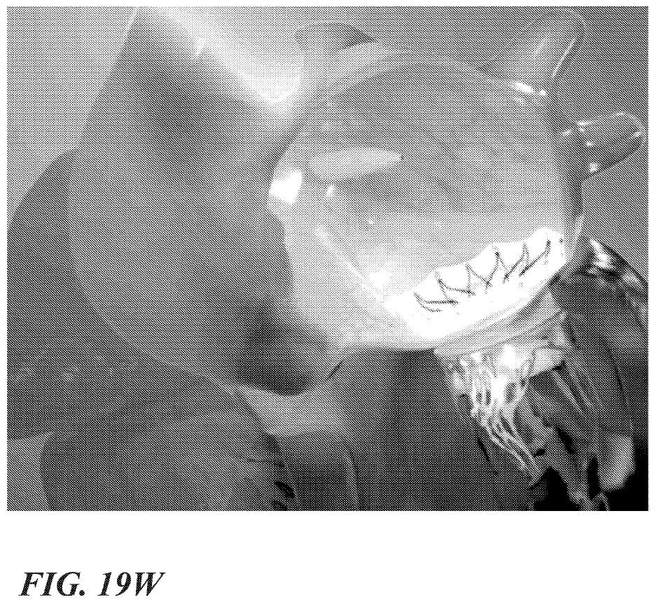

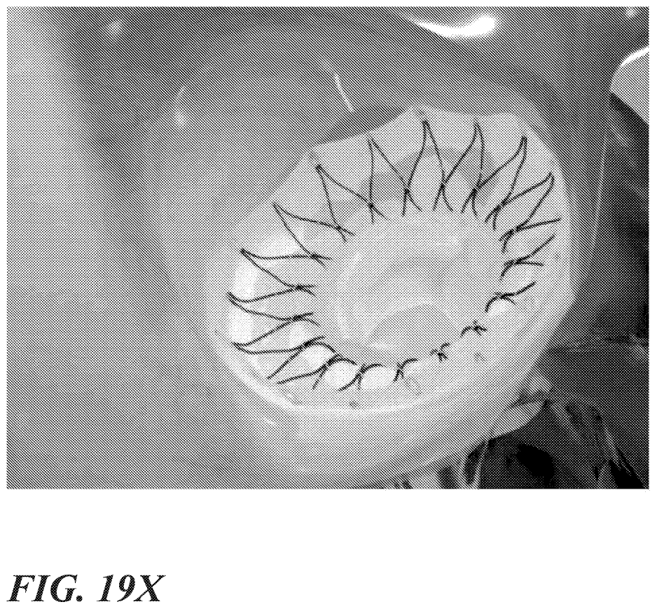

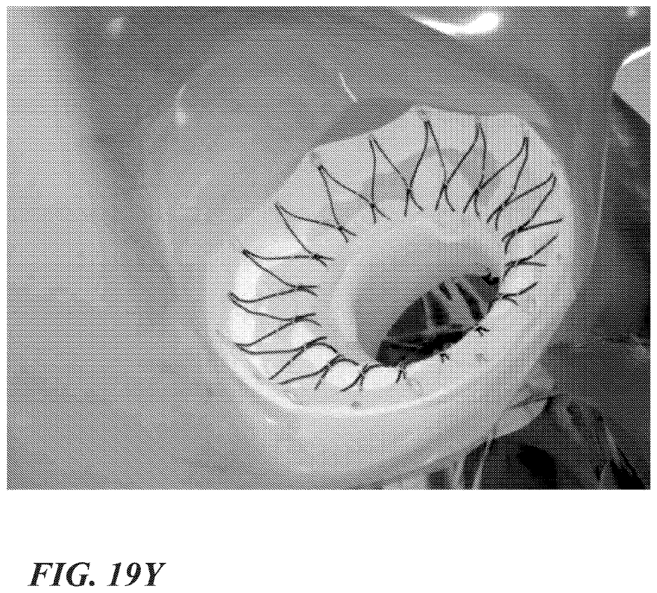

FIGS. 19A-19W illustrate systems and methods for delivering and implanting a prosthetic mitral valve and anchor within a heart of a subject. It should be understood that any of the valves, anchors, anchor delivery subsystems, and valve delivery subsystems described herein may be used in the methods described in FIGS. 19A-19W. As will be described below, the systems and methods provide a consistent, forgiving delivery procedure that can accommodate a range of patient anatomies, while fully addressing patient valve regurgitation without obstruction of LVOT. As will be apparent from the present disclosure the prosthetic mitral valve and systems and methods of delivery provide a number of clinical benefits and features over other systems on the market.

The valve delivery system of the present disclosure provides forgiving delivery of the anchor transeptally through the native valve and into the left ventricle. The clinician is given fine control of the position of the anchor, and therefore the shape of the guide arm, during encircling, allowing for adjustment of the guide arm radial position to ensure desired chords are captured. While the delivery system provides the ability to capture all of the chords in a single pass (e.g., from between 1 and up to 2.5 rotations of the guide arm), the delivery system also provides the ability to capture only some of the chords in a first revolution (e.g., 1 rotation) of the guide arm/anchor, and to capture the remaining chords on the subsequent revolutions of the guide arm/anchor (e.g., the remaining 1-1.5 rotations). The anchor delivery system provides rotation-based encircling with the ability to reverse and re-encircle the anchor if the clinician is unhappy with device placement or does not capture the desired anatomy within the anchor (e.g., the chords). Since the anchor is (e.g., wholly) contained within the guide arm of the delivery device during encircling, the clinician can easily reverse and re-encircle to safely fix the issue and continue the procedure without having to recapture a deployed anchor. The system is designed and configured to protect the anatomy from chordal injury/rupture.

In addition to being able to control the reach of the guide arm, the clinician is also given full independent control over the axial height of the anchor during and after encircling, as well as the rotational position of the anchor and guide arm. The delivery system and methods disclosed herein further facilitate determination of chordal capture. The position and orientation of the guide arm, and therefore the anchor (carried within), can be visualized with echo (ultrasound) alone during encircling and delivery. This provides for visualization of leaflets traveling outside the guide arm and/or direction visualization of chords. Biplane views can be fixed during procedure, so the clinician can check leaflet mobility throughout delivery. Visualization of the guide arm also allows for proper alignment of the anchor. The clinician can use the echo visualization to align the (e.g., distal portion of the) guide arm to be co-planar with the annulus. If the clinician achieves balanced capture of the chordae, the guide arm will remain coplanar with the annulus after encircling. An unbalanced or canted guide arm after encircling can indicate to the clinician that additional encircling or re-encircling is required.

In some embodiments, when the anchor is (e.g., fully) deployed from the delivery system into the heart, the anchor is completely released with no tether or other connection to other devices prior to valve deployment. The anchor is stably positioned by circumscribing and gently gathering chordae/leaflets in the left ventricle, while being completely free from (e.g., anchor) delivery system interaction once deployed. The inner diameter of the untethered anchor provides a target through which a guidewire is placed, with the valve delivery system advanced along the guidewire. All of the above features provide fine-tuned control of encircling device, and easy reversibility and safety and re-encircling without undue risk to patient tissue.

The prosthetic valve of the present disclosure also provides a number of advantages over competitors and clinical benefits to the patient. Importantly, the prosthetic valve is designed and configured to self-center within the target anatomy after deployment from the valve delivery system. The prosthetic valve is configured to self-center even with non-coaxial delivery or placement of the valve within the annulus and anchor. Coaxial delivery in this context refers to a central (longitudinal) axis of the prosthetic valve and a central axis of the anchor (e.g., axis perpendicular to the plane(s) containing the anchor). For example, the frame is tolerant to up to 45 degrees of off-axis delivery. The stiffness of the wide atrial brim enables this self-centering, balanced against the softness or compliance of the atrial flared portion to be atraumatic and prevent damage to tissue of the atrium and annulus. The short axial heigh of the ventricular flare or ventricular side of the prosthetic valve (e.g., less than 10 mm) allows the valve to deploy and self-center.

The prosthetic mitral valve of the present disclosure prevents paravalvular leaks (PVL) after implantation. The frame design, including the combination of a soft and wide atrial brim, a narrow central waist that interacts with the anchor to pinch inferior/superior to the annulus, and the fabric selection of the valve completely seals the valve against the anatomy reducing or eliminating the risk of blood flowing between the implanted valve and the cardiac tissue. Once the valve is implanted, is seated to the atrial floor with the wide atrial brim.

The prosthetic valve of the present disclosure is further designed and configured to reduce or limit left ventricular outflow tract obstruction (LVOTO). The short ventricular height of the valve (e.g., less than 10 mm), the self-centering nature of the valve (e.g., optimizing the angle of the valve with respect to the LVOT), and the tissue interaction between the valve and the anatomy (e.g., anterior leaflet capture/superior adjustment) all provide for a solution to LVOTO that has not been achieved with other competing devices. Specifically, the valve and anchor capture and pull the anterior leaflet away from the LVOT during expansion of the valve and axial adjustment of the anchor position, further reducing LVOTO.

Referring to FIG. 19A, an anchor delivery subsystem 30 including an outer steerable catheter 34 and an inner steerable catheter 36 can be navigated to the patient's right atrium and through the septum to the left atrium of the subject's heart. The anchor delivery subsystem 30 enables delivery of an anchor to encircle native tissue of the heart, as described in more detail below.

As shown in FIGS. 19B-19C, the guide arm 82 of the anchor delivery subsystem 30 can be advanced out of the distal end of the inner steerable catheter 36 into the left atrium. The active and passive portions of the guide arm 82, in combination with the anchor carried within, enable the guide arm to self-assemble to form a spiral shape (FIGS. 4-5) or a helical shape (FIGS. 6-7) in a left atrium. As previously described, the anchor also fully self-assembles and assumes it's at-rest shape within the guide arm when the guide arm is deployed in the left atrium.

In FIG. 19D, the inner steerable catheter 36 can be controlled to deflect the guide arm 82 towards the mitral annulus. In the illustrated embodiment, the spiral shape of the guide arm (e.g., the portion of the guide arm distal to bend 93 in FIG. 5) is generally parallel with the mitral annulus. In other embodiments where the guide arm has a helical shape, the planes of the helix distal to bend 93 are also generally parallel with the mitral annulus (e.g., the portions of the helical guide arm distal to bend 93 in FIG. 7).

In FIG. 19E, the guide arm 82 is counter-rotated and advanced to cross the mitral valve, through the leaflets, and into the left ventricle. Again it is noted that the guide arm is fully deployed and the anchor is fully assembled within the guide arm when the guide arm is advanced across the mitral valve. The planarity of the guide arm with respect to the mitral valve can be maintained as the guide arm crosses the mitral valve, as shown.

Referring to FIG. 19F, the encircling process can begin. As described above, the distal tip of the guide arm 82 can be extended radially outwards, for example by selected proximal retraction of the anchor therein. In the illustrated embodiment, the distal tip of the guide arm is extended toward the left ventricular outflow tract (LVOT). In FIG. 19G, the guide arm can be rotated to encircle chordae/leaflets within the left ventricle. Since the encircling process can be performed under echo imaging to provide visualization of the guide arm, the user can actively manipulate the distal tip of the guide arm to capture the desired chordae/leaflets. This may include extending the distal tip of the guide arm radially outwards or pulling the distal tip radially inwards depending on the patient specific anatomy.

In the example of FIG. 19H, the position of the guide arm has been assessed and a determination made that at least some chordae or leaflet tissue has not been correctly encircled. For repositioning the anchor delivery subsystem, the clinician can readily and independently adjust the axial height, circumferential (rotational) position, and/or radial (reach) position of the grabber arm to de-encircle/re-encircle, as often as desired. This delivery flexibility, along with the distinctive visualization characteristics of the anchor delivery catheter, provide the clinician with precise and repeatable control of encircling chordae.

At FIG. 19I, the guide arm is retracted or counter-rotated to partially de-encircle some portion of previously encircled chordae. The process of re-encircling is a simple procedure in which the user counter-rotates the guide arm (as in FIG. 191), and then re-encircles to fully capture the desired anatomy (FIGS. 19J-19K). Adjusting or changing the radial position of the distal tip of the guide arm can optionally be done at any point during encircling (rotating) or de-encircling (counter-rotating).

Once the chordae are determined to be satisfactorily (e.g., fully) encircled (FIG. 19L), the guide arm and the anchor delivery subsystem can be proximally retracted from the anchor without disturbing its position, the anchor delivery subsystem removed from the patient to leave only the anchor 88 in place within the left ventricle and surrounding the desired chordae/leaflets (FIG. 19M-19N). This is accomplished by maintaining the anchor position with the tether while withdrawing the guide arm into the inner steerable catheter, until the distal end of the guide arm clears the proximal end of the anchor and the anchor delivery subsystem is decoupled from the anchor. The inner steerable catheter is then pulled into the outer steerable catheter and the entire subsystem is removed from the subject. The result is the anchor 88 being deployed and anchored around chordae of the left ventricle with no connection to any other system component (e.g., no tether or other linkage is left behind when the anchor is deployed). Additionally, while the anchor is deployed around the chordae, the axial position of the anchor is not fixed to the anatomy. Therefore, the anchor can slide or be moved axially (e.g., towards or away from the annulus). This is described more fully below when self-expansion and positioning of the valve is described.

At FIG. 19O, the valve delivery can be initiated. First, a guidewire can be inserted through the septum, into the left atrium, through the mitral valve annulus, and through the anchor into the left ventricle.

In FIG. 19P, the valve delivery subsystem is advanced over the guidewire, through the septum, and into the left atrium. FIG. 19P shows the valve capsule 152 (containing the valve prosthesis) and the nose cone 150 in the left atrium. FIG. 19Q shows the valve capsule and nose cone advanced through the annulus with the nose cone positioned past the anchor and the valve capsule extending across/through the anchor.

In FIG. 19R, the valve capsule is partially retracted from the nose cone, to allow for (partial) self-expansion or assembly of the ventricular portion of the valve prosthesis. In some embodiments, the ventricular portion of the valve expands into the anchor. In other embodiments, referring to FIG. 19S, a physician may determine that the anchor and/or valve should be repositioned, and the valve delivery subsystem can be retracted or pulled towards the mitral annulus to capture the ventricular portion of the frame within the anchor. In some embodiments, the anchor can be lifted or pulled towards the annulus with the partially expanded valve by manipulation (e.g., proximal retraction) via the valve delivery subsystem, as indicated by the arrows. As described above, implanting the valve and anchor higher in the anatomy can help to reduce or prevent LVOTO. When the anchor is lifted or pulled towards annulus, the anterior leaflet can be captured and bunched up by the anchor into or against the annulus. This act of capturing the anterior leaflet, and pulling or bunching up the leaflet against the annulus moves tissues away from the LVOT, thereby reducing LVOTO.

In FIG. 19T, the valve capsule can be fully retracted to expose the atrial portion and atrial brim of the valve frame structure, allowing for self-expansion or the atrial portion within the left atrium. Once the atrial portion is fully expanded, the annulus is sealed with the wide and conformable atrial brim of the valve frame, the position of the atrial brim maintained and/or pressed downward by the anchor firmly positioned at the valve waist and capturing native tissues therebetween.

In FIG. 19U, the valve delivery subsystem is removed leaving the valve prosthesis implanted within the valve and anchor and sealing the mitral annulus. In some examples, the valve frame is self-expanded within the anchor such that the anchor rests on the ventricular flared portion of the valve frame. Continued self-expansion of the valve frame can cause an adjustment of the axial position of the helical anchor with respect to the frame. Referring to FIGS. 19U-19W, self-expansion of the frame when the anchor is positioned on the flared ventricular portion can cause the anchor to move axially along the frame towards the central waist portion of the frame (e.g., towards the native valve). Similarly, this axial movement of the anchor along the frame can also pull the atrial flared portion of the valve frame into contact with atrial tissue adjacent to the native valve, thereby sealing the valve frame against the tissue. Therefore, the axial movement of the anchor on the frame when the frame is self-expanding serves two purposes: 1) pulling the valve frame into a sealing configuration with the tissue, and 2) self-centering the anchor on the central waist portion of the frame.

When the anchoring device is deployed around the replacement valve frame, the anchoring device is configured to apply a retention force no larger than 1.5 lbs to the mitral valve replacement frame to retain the mitral valve replacement frame within a mitral annulus of the subject. In some examples, the anchoring device is configured to apply a retention force of 4 lbs or less to the mitral valve replacement frame to retain the valve in the desired location within the anatomy. In some embodiments, the retention force is between 1.5 lbs and less than 4 lbs. The retaining force applied by the anchor against the frame is much lower compared to competing devices on the market due to the unique shape of the frame (e.g., the flared atrial portion, the central waist portion, and the flared ventricular portion).

In some embodiments, the retention force of no larger than 1.5 lbs., or alternatively the retention force of less than 4 lbs. or between 1.5 lbs. and 4 lbs., is defined as the force applied by the anchor to the valve frame when the anchor is positioned within or engaged with the central waist portion of the valve frame (e.g., when the valve is under insignificant axial loads). Generally, when the anchor is positioned within or against the central waist portion of the valve frame, the retention forces are very low, e.g., no more than 1.5 lbs. or alternatively, less than 4 lbs. of pressure. However, it should be understood that significant axial loads applied to the valve frame (e.g., at higher heart pressures during the heart cycle) can cause the frame to move axially within the anatomy. This causes the position of the anchor relative to the frame to change, causing the anchor to “ride up” or move away from the central waist portion towards the flared atrial portion or the flared ventricular portion. As the anchor engages with these flared portions of the valve frame (e.g., when the system is under axial load), the interaction of the anchor with the flared valve portions increases the retention forces, for example, up to 10 lbs of force or more. This dynamic interplay between the anchor and the valve frame allows for the system to self-correct when large axial loads are applied.

To summarize, when the valve frame and anchor are in an “at-rest” configuration which can be defined as a situation in which insignificant axial forces are applied and therefore the anchor resides within or against the central waist portion of the frame, then the retention forces applied by the anchor to the frame are very low (e.g., no larger than 1.5 lbs., between 1.5 lbs. and 4 lbs., or less than 4 lbs.). However, when large axial loads are applied to the valve frame and anchor, causing the anchor to engage with or contact the flared portions of the valve frame, the retention forces applied by the anchor to the frame increase, which results in the anchor self-correcting or moving back towards the at-rest state in which the anchor resides within the central waist portion. This dynamic interplay ensures that the valve frame and anchor remain positioned in the desired location within the anatomy.

Additionally, the retaining force is low because of the ability of the anchoring device to change both in diameter and number of turns when the frame is self-expanded within the anchor. The system described herein uniquely allows for the diameter of the anchoring device to expand when the frame is self-expanded within the anchor. Similarly, the system described herein uniquely allows for the number of turns of the anchoring device to reduce when the frame is self-expanded within the anchor. Furthermore, an axial position of the anchoring device can be self-adjusted when the frame is self-expanded to allow for the anchor to engage with the central waist portion, which advantageously seals the frame against the desired anatomy.

FIGS. 19X-19Y illustrate a left-atrial view of the valve frame prosthesis implanted within the mitral annulus with the leaflets closed (FIG. 19X) and opened (FIG. 19Y).

The valve delivery subsystem is a low profile valve delivery system that allows control of valve position until the very end of the delivery procedure. The valve delivery subsystem is a true 28 Fr delivery profile with steerability that allows for familiar and easy positioning of the valve frame in the target location. The valve delivery subsystem allows for deployment of the collapsed or compressed valve frame within an already deployed anchor. Expansion of the valve frame structure captures the anchor and controls the final anchor position. Emphasis has been provided above that it is desirable to have the anchor deployed in a “high” position towards the left atrium so as to avoid LVOTO. While the anchor position can be controlled with the anchor delivery subsystem, it should also be understood that the anchor position can be adjusted or pulled upwards with the valve delivery subsystem after the valve has been allowed to expand within the anchor.

This disclosure provides details around a forgiving mitral valve replacement procedure and system specifically designed for the mitral anatomy. The systems and methods disclosed herein solve for an unmet need by providing a delivery system and delivery procedure that is familiar to physicians with a small learning curve, an implant that is adaptable and applicable to all anatomies, and an implant that reliable eliminates mitral regurgitation (MR) without the risk of complications associated with other mitral valve replacement devices on the market.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected,” “attached” or “coupled” to another feature or element, it can be directly connected, attached or coupled to the other feature or element or intervening features or elements may be present. In contrast, when a feature or element is referred to as being “directly connected,” “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise,” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

### METHODS OF USE

In operation, a prosthetic valve system according to embodiments of the present disclosure is delivered and implanted as follows. An anchor delivery catheter that carries the anchor in a delivery configuration is advanced transeptally to the left atrium. The distal portion (e.g., guide arm) of the anchor delivery catheter, along with the anchor contained therein, self-assemble into an encircling configuration. The anchor delivery catheter is advanced through the valve into the ventricle of the heart. The clinician is provided with independent control of rotation, radial extent (“reach”), and axial position (“height”) of the guide arm to encircle native tissue (e.g., leaflets and/or chordae). Upon determination of satisfactory encircling, the anchor delivery catheter is removed and the anchor is left in place (e.g., solely in the ventricle) with the encircled tissue contained (e.g., radially) therein. In some embodiments, the anchor is (e.g., fully) deployed from the delivery system into the heart, the anchor is completely released with no tether or other connection to other devices prior to valve deployment. The anchor is stably positioned by circumscribing and gently gathering chordae/leaflets in the ventricle, while being completely free from (e.g., anchor) delivery system interaction once deployed. The inner diameter of the untethered anchor provides a target through which a guidewire is placed, with the valve delivery system advanced along the guidewire. All of the above features provide fine-tuned control of encircling device, and easy reversibility and safety and re-encircling without undue risk to patient tissue. The valve is deployed in a stepwise or piecewise manner. For example, first the ventricular portion of the frame is expanded into the anchor, allowing engagement between the outflow and/or central waist portion of the frame and the anchor. This allows the clinician to “pull” or raise the anchor (and optionally the valve) up into or towards the annulus. Then the atrial side of the frame is self-expanded, firmly fixing the prosthetic valve into the anatomy in combination with the anchor positioned about a waist of the prosthetic valve. The open (free) ends of the anchor enable it to resize as the self-expanding valve is expanded therein. The stiffness of the anchor in its (e.g., initial) deployed state is selected to be such that the expansion force of the self-expanding valve can modify the (e.g., radial) dimension of the anchor to ensure the valve achieves its target size. For example, the radial dimension of the deployed anchor can increase to accommodate and conform to the self-expanded valve perimeter. Further, the anchor interaction with the expanding valve is such that, during partial deployment of the valve, an axial position of the anchor with respect to the valve body (and/or the native anatomy) can be altered, with no appreciable friction or resistance to the valve body and/or native anatomy. Upon full expansion of the valve into the anchor and anatomy, the anchor is securely coupled to firmly hold its position and maintain chordae/leaflets containment (e.g., encircling) against the valve frame. Separating the anchor delivery from the frame delivery provides a purpose-built system for mitral replacement, achieving a true 28F delivery system.

1. A mitral valve replacement system, comprising:
a helical anchoring device; and
a heart valve replacement frame configured to self-expand within the helical anchoring device;
wherein self-expansion of the heart valve replacement frame into the helical anchoring device is configured to reduce a number of turns of the helical anchoring device.

2. The system of claim 1, wherein self-expansion of the mitral valve replacement frame into the helical anchoring device reduces a number of turns of the helical anchoring device by up to 30%.

3. The system of claim 1, wherein self-expansion of the mitral valve replacement frame into the helical anchoring device reduces a number of turns of the helical anchoring device by 20% to 30%.

4. The system of claim 1, wherein the helical anchoring device comprises approximately 1.6 to 1.8 turns when it is deployed within the left ventricle.

5. The system of claim 4, wherein the helical anchoring device comprises approximately 1.1 to 1.25 turns when the mitral valve replacement frame is self-expanded within the helical anchoring device.

6. The system of claim 1, wherein the helical anchoring device is configured to be deployed entirely in a left ventricle of a human subject.

7. The system of claim 6, wherein the heart valve replacement frame comprises a flared atrial portion configured to engage atrial tissue of the subject, a narrowed central portion configured to engage the helical anchoring device, and a flared ventricular portion configured to extend into the left ventricle of the subject.

8. The system of claim 1, wherein a percentage of the helical anchor that overlaps with an adjacent turn of the helical anchor is reduced when the heart valve replacement frame is self-expanded into the helical anchoring device.

9. The system of claim 1, wherein the heart valve replacement frame comprises a flared ventricular portion, a central waist portion, and a flared atrial portion.

10. The system of claim 9, wherein the helical anchoring device is configured to reside within the central waist portion of the heart valve replacement frame when the heart valve replacement frame is self-expanded into the helical anchoring device

11. A mitral valve replacement system, comprising:
a helical anchoring device comprising configured to be disposed entirely within a left ventricle of a subject; and
a self-expandable mitral valve replacement frame comprising a flared atrial portion configured to engage atrial tissue of the subject, a narrowed central portion configured to engage the helical anchoring device, and a flared ventricular portion configured to extend into the left ventricle of the subject;
wherein self-expansion of the mitral valve replacement frame into the helical anchoring device is configured to reduce a number of turns of the helical anchoring device.

12. The system of claim 11, wherein self-expansion of the mitral valve replacement frame into the helical anchoring device reduces a number of turns of the helical anchoring device by up to 30%.

13. The system of claim 11, wherein self-expansion of the mitral valve replacement frame into the helical anchoring device reduces a number of turns of the helical anchoring device by 20% to 30%.

14. The system of claim 11, wherein the helical anchoring device comprises approximately 1.6 to 1.8 turns when it is deployed within the left ventricle.

15. The system of claim 14, wherein the helical anchoring device comprises approximately 1.1 to 1.25 turns when the mitral valve replacement frame is self-expanded within the helical anchoring device.

16. The system of claim 11, wherein the helical anchoring device is configured to be deployed entirely in a left ventricle of a human subject.

17. The system of claim 16, wherein the heart valve replacement frame comprises a flared atrial portion configured to engage atrial tissue of the subject, a narrowed central portion configured to engage the helical anchoring device, and a flared ventricular portion configured to extend into the left ventricle of the subject.

18. The system of claim 11, wherein a percentage of the helical anchor that overlaps with an adjacent turn of the helical anchor is reduced when the heart valve replacement frame is self-expanded into the helical anchoring device.

19. The system of claim 11, wherein the helical anchoring device is configured to apply a retention force less than 4 lbs. to the valve replacement frame in an at-rest state in which the helical anchoring device resides within the narrowed central portion.

20. A method, comprising:
deploying a helical anchoring device entirely in a left ventricle of a subject;
advancing a mitral valve replacement frame through the helical anchoring device; and
self-expanding the mitral valve replacement device within the helical anchoring device to reduce a number of turns of the helical anchoring device.

21.-62. (canceled)
